(19) United States
(12) Patent Application Publication
(10) Pub. No.: US 2025/0215187 A1
(43) Pub. Date: Jul. 3, 2025

(54) IMPROVED METHOD FOR DEPOLYMERISING POLYETHYLENE TEREPHTHALATE

(71) Applicant: EVONIK OPERATIONS GMBH, Essen (DE)

(72) Inventors: Philip Heinrich REINSBERG, Rheinbach (DE); Michael HORN, Niederkassel (DE); Johannes RUWWE, Hanau (DE); Marc WEINER, Troisdorf (DE)

(73) Assignee: EVONIK OPERATIONS GMBH, Essen (DE)

(21) Appl. No.: 18/853,148

(22) PCT Filed: Nov. 18, 2022
(86) PCT No.: PCT/EP2022/082364

§ 371 Date: Oct. 1, 2024

(30) Foreign Application Priority Data
Apr. 4, 2022 (EP) 22166553.2

(51) Int. Cl. C08J 11/26; C08G 63/183; C08G 63/86; C25B 3/07; C25B 3/25; C25B 9/19; C25B 13/07; C25B 15/08

(52) U.S. Cl. CPC C08J 11/26; C08G 63/183; C08G 63/866; C25B 3/07; C25B 3/25; C25B 9/19; C25B 13/07; C25B 15/081; C08J 2367/03

(57) ABSTRACT

The invention relates to a method for depolymerising polyethylene terephthalate (“PET”), in which PET is reacted with electrolytically prepared alkali metal glycolate, in particular sodium or potassium glycolate, to form a mixture M1 comprising bis(2-hydroxyethyl) terephthalate (“BHET”). The method according to the invention is characterised in that BHET accounts for a particularly high proportion of the breakdown products in the mixture M1. As a result, the method according to the invention provides a high yield of BHET, which can be used directly for renewed PET production. The present invention also relates to a method for recycling PET, in which the BHET obtained in the method for depolymerising PET is polymerised again to PET, optionally after further purification from M1.

The present invention relates to a method of depolymerization of polyethylene terephthalate (=“PET”), in which PET is reacted with electrolytically produced alkali metal glycolate, especially sodium glycolate or potassium glycolate, to give a mixture M₁ comprising bis-2-hydroxyethyl terephthalate (=“BHET”; CAS No.: 959-26-2).

It is a feature of the method according to the invention that BHET forms a particularly high proportion among the cleavage products in the mixture M₁. As a result, the method according to the invention affords a high yield of BHET, which can be used directly for new production of PET.

The present invention thus also relates to a method of recycling PET, in which the BHET obtained in the method of depolymerization of PET, optionally after further purification from M₁, is polymerized again to give PET.

### BACKGROUND OF THE INVENTION

Polyethylene terephthalate (=“PET”) is one of the most important plastics which is used in textile fibres, as films, and as material for plastic bottles. In 2007 alone, the volume used in plastic bottles was ˜10⁷ t (W. Caseri, Polyethylenterephthalate, RD-16-03258 (2009) in F. Böckler, B. Dill, G. Eisenbrand, F. Faupel, B. Fugmann, T. Gamse, R. Matissek, G. Pohnert, A. Rühling, S. Schmidt, G. Sprenger, ROMPP [Online], Stuttgart, Georg Thieme Verlag, January 2022).

On account of its persistence and the volumes of refuse originating from PET, it constitutes one of the greatest environmental challenges at present. The solution to this problem lies in the avoidance and in the efficient reutilization of PET.

The prior art proposes multiple methods of cleavage of PET.

GB 784,248 A describes the methanolysis of PET.

Hydrolytic methods for depolymerization of PET are described by JP 2000-309663 A, U.S. Pat. No. 4,355,175 A and T. Yoshioka, N. Okayama, A. Okuwaki, Ind. Eng. Chem. Res. 1998, 37, 336-340. The reaction of PET with glycol is described in EP 0723951 A1, U.S. Pat. No. 3,222,299 A, WO 2020/002999 A2, by S. R. Shukla, A. M. Harad, Journal of Applied Polymer Science 2005, 97, 513-517 (“Shukla & Harad” hereinafter) and by N. D. Pingale, S. R. Shukla, European Polymer Journal 2008, 44, 4151-4156.

Shukla & Harad state that PET glycolysis gives rise to bis-2-hydroxyethyl terephthalate (=“BHET”). This cleavage product may simultaneously be used as reactant for production of new PET.

There is accordingly an interest in methods of depolymerization of PET in which a maximum proportion of BHET is obtained among the cleavage products.

The problem addressed by the present invention was that of providing such a method.

### BRIEF DESCRIPTION OF THE INVENTION

A method that solves the problem addressed by the invention has now surprisingly been found.

The present invention relates to a method of depolymerization of polyethylene terephthalate PET, comprising the following steps:
(a) producing a solution L₁<21> of M_{A} glycolate in glycol, where M_{A} is an alkali metal cation, especially selected from lithium, potassium, sodium, preferably selected from potassium, sodium, and most preferably sodium, in an electrolysis cell E<1> comprising
   at least one anode chamber K_{A}<11> having at least one inlet Z_{KA}<110>, at least one outlet A_{KA}<111>, and an interior I_{KA}<112> comprising an anodic electrode E_{A}<113>,
   at least one cathode chamber K_{K}<12> having at least one inlet Z_{KK}<120>, at least one outlet A_{KK}<121>, and an interior I_{KK}<122> comprising a cathodic electrode E_{K}<123>,
   and optionally at least one interposed middle chamber K_{M}<13> having at least one inlet Z_{KM}<130>, at least one outlet A_{KM}<131> and an interior I_{KM}<132>, where I_{KA}<112> and I_{KM}<132> are then divided from one another by a diffusion barrier D<14>, and A_{KM}<131> is connected by a connection V_{AM}<15> to the inlet Z_{KA}<110>, such that liquid can be passed from I_{KM}<132> into I_{KA}<112> via the connection V_{AM}<15>,
where
   in the cases in which the electrolysis cell E<1> does not comprise a middle chamber K_{M}<13>, I_{KA}<112> and I_{KK}<122> are divided from one another by a dividing wall W<16>,
   in the cases in which the electrolysis cell E<1> comprises at least one middle chamber K_{M}<13>, I_{KK}<122> and I_{KM}<132> are divided from one another by a dividing wall W<16>,
wherein the dividing wall W<16> has one side S_{KK}<161> having the surface O_{KK}<163> and a side S_{A/MK}<162> which is on the opposite side from S_{KK}<161> and has the surface O_{A/MK}<164>, wherein the dividing wall W<16> comprises at least one alkali metal cation-conducting solid-state electrolyte ceramic F_{A}<18> in such a way that the alkali metal cation-conducting solid-state electrolyte ceramic F_{A}<18> encompassed by the dividing wall W<16> makes direct contact with the interior I_{KK}<122> on the S_{KK}<161> side via the surface O_{KK}<163>,
and wherein
   in the cases in which the electrolysis cell E<1> does not comprise a middle chamber K_{M}<13>, the alkali metal cation-conducting solid-state electrolyte ceramic F_{A}<18> encompassed by the dividing wall W<16> makes direct contact with the interior I_{KA}<112> on the S_{A/MK}<162> side via the surface O_{A/MK}<164>,
   in the cases in which the electrolysis cell E<1> comprises at least one middle chamber K_{M}<13>, the alkali metal cation-conducting solid-state electrolyte ceramic F_{A}<18> encompassed by the dividing wall W<16> makes direct contact with the interior I_{KM}<132> on the S_{A/MK}<162> side via the surface O_{A/MK}<164>,
(α) wherein, in the electrolysis cell E<1>, when it does not comprise a middle chamber K_{M}<13>, the following steps (α1), (α2), (α3) that proceed simultaneously are performed:
   (α1) a solution L₂<22> comprising glycol is directed through I_{KK}<122>,
   (α2) a neutral or alkaline, aqueous solution L₃<23> of a salt S comprising M_{A} as cation is directed through I_{KA}<112>,
   (α3) voltage is applied between E_{A}<113> and E_{K}<123>, or
(β) wherein, in the electrolysis cell E<1>, when it comprises at least one middle chamber K_{M}<13>, the following steps (β1), (β2), (β3) that proceed simultaneously are performed:
   (β1) a solution L₂<22> comprising glycol is directed through I_{KK}<122>,
   (β2) a neutral or alkaline, aqueous solution L₃<23> of a salt S comprising M_{A} as cation is directed through I_{KM}<132>, then via V_{AM}<15>, then through I_{KA}<112>,
   (β3) voltage is applied between E_{A}<113> and E_{K}<123>,
which affords the solution L₁<21> at the outlet A_{KK}<121>, the concentration of M_{A} glycolate being higher in L₁<21> than in L₂<22>,
and which affords an aqueous solution L₄<24> of S at the outlet A_{KA}<111>, the concentration of S being lower in L₄<24> than in L₃<23>;
(b) reacting the solution L₁<21> with PET to give a mixture M₁ comprising bis-2-hydroxyethyl terephthalate (=“BHET”).

In a further aspect, the present invention relates to a method of recycling PET, in which the BHET obtained in the method according to the invention for depolymerization is polymerized in a step (2) to give PET.

It has been found that, surprisingly, the reaction of the PET with the solution L₁<21> obtained by the electrolytic method according to the invention affords a higher proportion of BHET than in conventional methods in which the alkaline alkali metal glycolate solution is obtained by mixing the glycol in the corresponding alkali metal hydroxide.

### FIGURES

FIGS. 1 A and 1 B

FIG. 1 A (=“FIG. 1 A”) shows the method according to the invention for production of the sodium glycolate solution L₁<21> in an electrolysis cell E<1>. This comprises a cathode chamber K_{K}<12> and an anode chamber K_{A}<11>.

The cathode chamber K_{K}<12> comprises a cathodic electrode E_{K}<123> in the interior I_{KK}<122>, an inlet Z_{KK}<120> and an outlet A_{KK}<121>.

The anode chamber K_{A}<11> comprises an anodic electrode E_{A}<113> in the interior I_{KA}<112>, an inlet Z_{KA}<110> and an outlet A_{KA}<111>.

The two chambers K_{A}<11> and K_{K}<12> are bounded by an outer wall W_{A}<80> of the two-chamber cell E<1>. The interior I_{KK}<122> is also divided from the interior I_{KA}<112> by a dividing wall W<16> consisting of a sheet of an NaSICON solid-state electrolyte ceramic F_{A}<18> which is selectively permeable to sodium ions. The NaSICON solid-state electrolyte ceramic F_{A}<18> extends over the entire depth and height of the two-chamber cell E<1>. The dividing wall has two sides S_{KK}<161> and S_{A/MK}<162>, the surfaces O_{KK}<163> and O_{A/MK}<164> of which contact the respective interior I_{KK}<122> or I_{KA}<112>.

A pH 10.5 aqueous solution of sodium chloride L₃<23> is introduced via the inlet Z_{KA}<110>, counter to the direction of gravity, into the interior I_{KA}<112>.

A solution of 1% by weight of sodium glycolate in glycol L₂<22> is directed into the interior I_{KK}<122> via the inlet Z_{KK}<120>.

At the same time, a voltage is applied between the cathodic electrode E_{K}<123> and the anodic electrode E_{A}<113>. As a result, in the interior I_{KK}<122>, glycol in the electrolyte L₂<22> is reduced to glycolate and H₂ (HOCH₂CH₂OH+e⁻→HOCH₂CH₂O⁻+½ H₂; and additionally also HOCH₂CH₂O⁻+e⁻→⁻OCH₂CH₂O⁻+½ H₂). At the same time, sodium ions diffuse from the interior I_{KA}<112> through the NaSICON solid-state electrolyte ceramic F_{A}<18> into the interior I_{KK}<122>. Overall, this increases the concentration of sodium glycolate in the interior I_{KK}<122>, which affords a glycolic solution of sodium glycolate L₁<21> having an elevated sodium glycolate concentration compared to L₂<22>, of ˜ 20% by weight of Na glycolate in glycol, at the outlet A_{KK}<121>.

In the interior I_{KA}<112>, the oxidation of chloride ions takes place to give molecular chlorine (Cl⁻→½ Cl₂+e⁻). In the outlet A_{KA}<111>, an aqueous solution L₄<24> is obtained, in which the content of NaCl is lower compared to L₃<23>. Chlorine gas (Cl₂) in water, according to the reaction Cl₂+H₂O→HOCl+HCl, forms hypochlorous acid and hydrochloric acid, which give an acidic reaction with further water molecules. The acidity damages the NaSICON solid-state electrolyte ceramic F_{A}<18>.

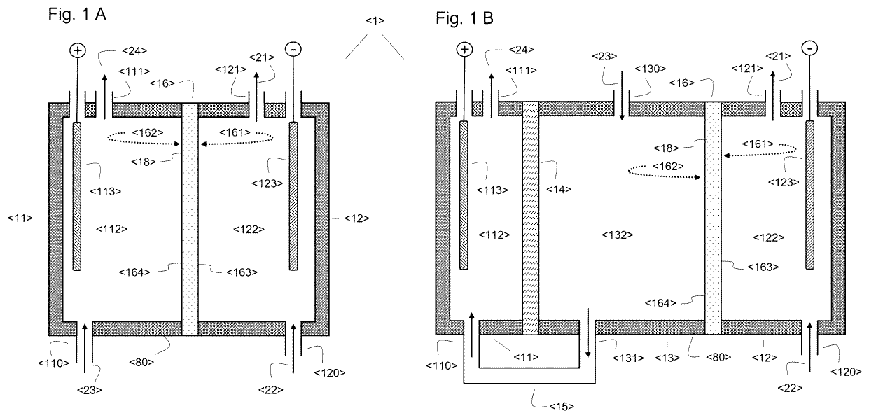

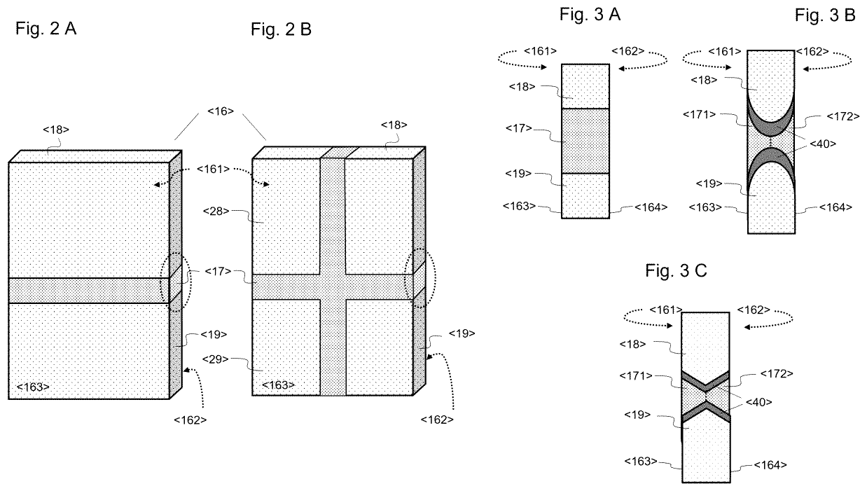

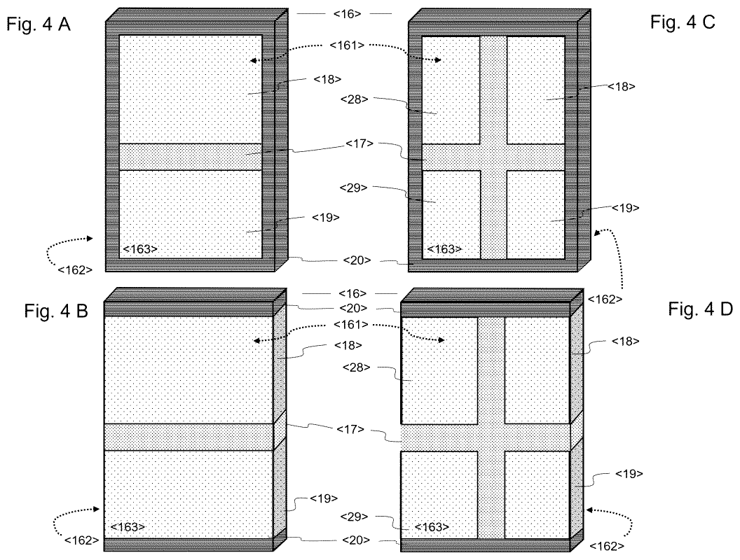

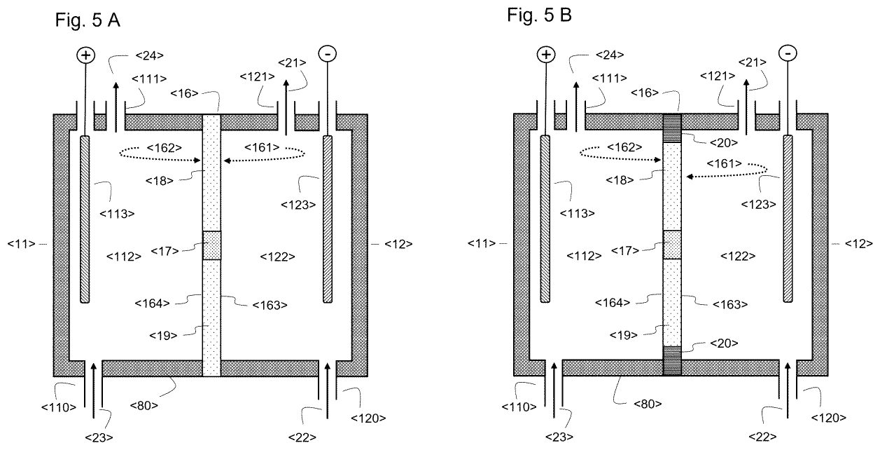

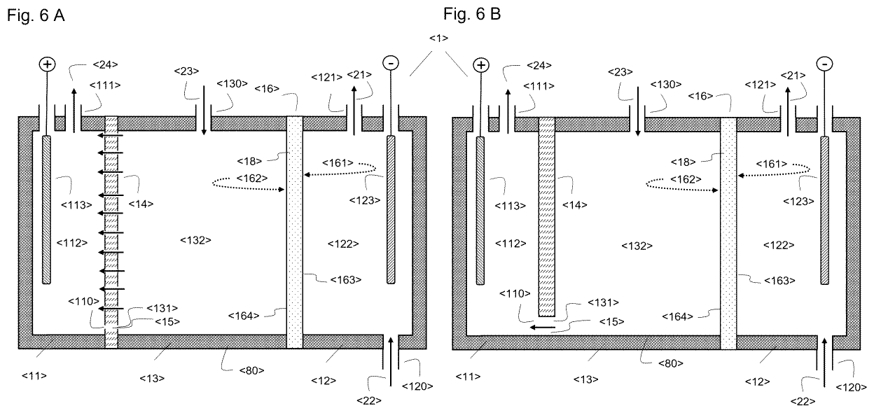

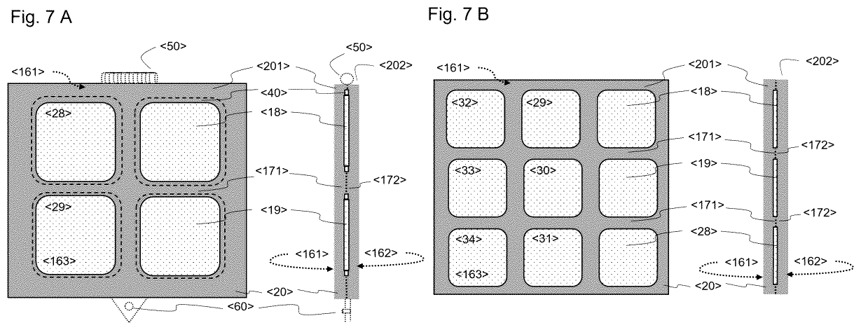

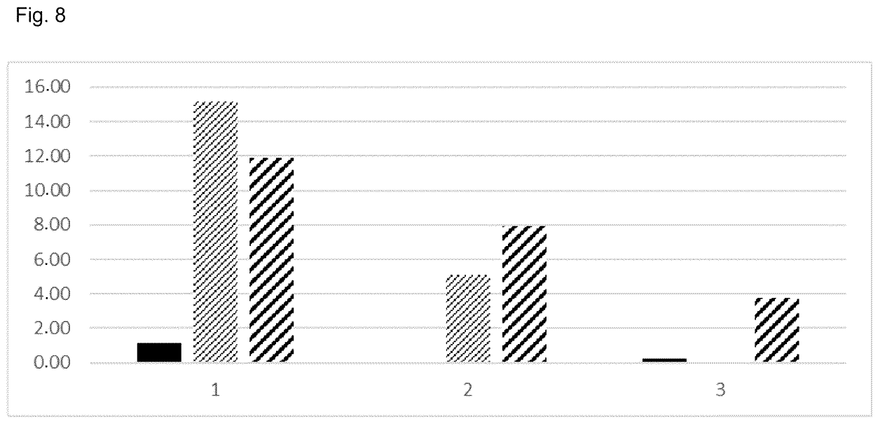

FIG. 1 B (=“FIG. 1 B”) shows a further embodiment of the method according to the invention in the form of an electrolysis cell E<1> comprising a middle chamber K_{M}<13>. This three-chamber cell E<1> accordingly comprises a cathode chamber K_{K}<12>, an anode chamber K_{A}<11> and an interposed middle chamber K_{M}<13>.

The cathode chamber K_{K}<12> comprises a cathodic electrode E_{K}<123> in the interior I_{KK}<122>, an inlet Z_{KK}<120> and an outlet A_{KK}<121>.

The anode chamber K_{A}<11> comprises an anodic electrode E_{A}<113> in the interior I_{KA}<112>, an inlet Z_{KA}<110> and an outlet A_{KA}<111>.

The middle chamber K_{M}<13> comprises an interior I_{KM}<132>, an inlet Z_{KM}<130> and an outlet A_{KM}<131>.

The interior I_{KA}<112> is connected to the interior I_{KM}<132> via the connection V_{AM}<15>.

The three chambers are bounded by an outer wall W_{A}<80> of the three-chamber cell E<1>. The interior I_{KM}<132> of the middle chamber K_{M}<13> is also divided from the interior I_{KK}<122> of the cathode chamber K_{K}<12> by a dividing wall W<16> consisting of a sheet of a NaSICON solid-state electrolyte ceramic F_{A}<18> which is selectively permeable to sodium ions. The NaSICON solid-state electrolyte ceramic F_{A}<18> extends over the entire depth and height of the three-chamber cell E<1>. The dividing wall has two sides S_{KK}<161> and S_{A/MK}<162>, the surfaces O_{KK}<163> and O_{A/MK}<164> of which contact the respective interior I_{KK}<122> or I_{KM}<132>.

The interior I_{KM}<132> of the middle chamber K_{M}<13> is additionally divided in turn from the interior I_{KA}<112> of the anode chamber K_{A}<11> by a diffusion barrier D<14>. The NaSICON solid-state electrolyte ceramic F_{A}<18> and the diffusion barrier D<14> extend over the entire depth and height of the three-chamber cell E<1>. The diffusion barrier D<14> is a cation exchange membrane (sulfonated PTFE).

In the embodiment according to FIG. 1 B, the connection V_{AM}<15> is formed outside the electrolysis cell E<1>, especially by a tube or hose, the material of which may be selected from rubber, metal and plastic. The connection V_{AM}<15> can guide liquid from the interior I_{KM}<132> of the middle chamber K_{M}<13> into the interior I_{KA}<112> of the anode chamber K_{A}<11> outside the outer wall W_{A}<80> of the three-chamber cell E<1>. The connection V_{AM}<15> connects the outlet A_{KM}<131> that penetrates the outer wall W_{A}<80> of the electrolysis cell E<1> at the base of the middle chamber K_{M}<13> to the inlet Z_{KA}<110> that penetrates the outer wall W_{A}<80> of the electrolysis cell E<1> at the base of the anode chamber K_{A}<11>.

A pH 10.5 aqueous solution of sodium chloride L₃<23> is introduced via the inlet Z_{KM}<130>, in the direction of gravity, into the interior I_{KM}<132> of the middle chamber K_{M}. The connection V_{AM}<15> connects the interior I_{KM}<132> of the middle chamber K_{M}<13> to the interior I_{KA}<112> of the anode chamber K_{A}<11>. Sodium chloride solution L₃<23> is directed through this connection V_{AM}<15> from the interior I_{KM}<132> into the interior I_{KM}<112>.

A solution of ˜1% by weight of sodium glycolate in glycol L₂<22> is directed into the interior I_{KK}<122> via the inlet Z_{KK}<120>.

At the same time, a voltage is applied between the cathodic electrode E_{K}<123> and the anodic electrode E_{A}<113>. As a result, in the interior I_{KK}<122>, glycol in the electrolyte L₂<22> is reduced to glycolate and H₂ (HOCH₂CH₂OH+e⁻→HOCH₂CH₂O⁻+½ H₂; and additionally also HOCH₂CH₂O⁻+e⁻→OCH₂CH₂O⁻+½ H₂). At the same time, sodium ions diffuse from the interior I_{KM}<132> of the middle chamber K_{M}<103> through the NaSICON solid-state electrolyte ceramic F_{A}<18> into the interior I_{KK}<122>. Overall, this increases the concentration of sodium glycolate in the interior I_{KK}<122>, which affords a glycolic solution of sodium glycolate L₁<21> having an elevated sodium glycolate concentration compared to L₂<22>, of ˜ 20% by weight of sodium glycolate in glycol, at the outlet A_{KK}<121>.

In the interior I_{KA}<112>, the oxidation of chloride ions takes place to give molecular chlorine (Cl⁻→½ Cl₂+e⁻). In the outlet A_{KA}<111>, an aqueous solution L₄<24> is obtained, in which the content of NaCl is lower compared to L₃<23>. Chlorine gas (Cl₂) in water, according to the reaction Cl₂+H₂O→HOCl+HCl, forms hypochlorous acid and hydrochloric acid, which give an acidic reaction with further water molecules. The acidity would damage the NaSICON solid-state electrolyte ceramic F_{A}<18>, but is restricted to the anode chamber K_{A}<11> by the arrangement in the three-chamber cell, and hence kept away from the NaSICON solid-state electrolyte ceramic F_{A}<18> in the electrolysis cell E. This considerably increases the lifetime thereof.

FIGS. 2 A and 2 B

FIG. 2 A (=“FIG. 2 A”) shows a preferred dividing wall W<16>. This comprises two NaSICON solid-state electrolyte ceramics F_{A}<18> and F_{B}<19> that are separated from one another by a separating element T<17> and are each secured thereto in a gapless manner. The separating element T<17> has the geometric shape of a cuboid, on the opposite sides of which F_{A}<18> and F_{B}<19> are secured in a gapless manner (for example by adhesive).

The side S_{KK}<161> with the surface O_{KK}<163> lies in the plane of the drawing, and the side S_{A/MK}<162> with the surface O_{A/MK}<164> not visible in FIG. 2 A behind the plane of the drawing.

FIG. 2 B (=“FIG. 2 B”) shows another embodiment of a preferred dividing wall W<16>. This comprises four NaSICON solid-state electrolyte ceramics F_{A}<18>, F_{B}<19>, F_{C}<28>, F_{D}<29> that are separated from one another by a separating element T<17> and are each secured thereto in a gapless manner. The separating element T<17> has the shape of a cross, on the opposite sides of which F_{A}<18>, F_{B}<19>, F_{C}<28> and F_{D}<29> are fixed.

The side S_{KK}<161> with the surface O_{KK}<163> lies in the plane of the drawing, and the side S_{A/MK}<162> with the surface O_{A/MK}<164> not visible in FIG. 2 B behind the plane of the drawing.

FIGS. 3 A to 3 C

FIG. 3 A (=“FIG. 3 A”) shows the detail view indicated by a dotted circle in FIGS. 2 A and 2 B. As described, the respective solid-state electrolyte ceramics F_{A}<18> and F_{B}<19> are secured to the separating element T<17>, for example by adhesive.

FIG. 3 B (=“FIG. 3 B”) illustrates a further embodiment of a preferred dividing wall W. Here, the separating element T<17> has two concave depressions (grooves) into which the two solid-state electrolyte ceramics F_{A}<18> and F_{B}<19> are fitted. For this purpose, the shape of the edges of the solid-state electrolyte ceramics F_{A}<18> and F_{B}<19> can be correspondingly mechanically adjusted. In addition, a seal Di<40> is used, which is mounted, for example, with an adhesive on the separating element T<17> and the respective solid-state electrolyte ceramic F_{A}<18> or F_{B}<19>. The separating element T<17> may consist here of two or more parts <171> and <172>, which, as indicated by the dotted line in FIG. 3 B, may be secured to one another. In the case of an appropriate geometry and fit of the shape of the edges of the solid-state electrolyte ceramics F_{A}<18> and F_{B}<19>, the latter may be clamped between the two parts <171> and <172>, which further improves the stability of the connection of separating element T<17>/ceramic F_{A}<18> or F_{B}<19> and the integrity of the dividing wall W<16>.

FIG. 3 C (=“FIG. 3 C”) illustrates a further embodiment of a preferred dividing wall W. This corresponds to that described in FIG. 3 B, except that the depressions (grooves) in the separating element T<17> into which the two solid-state electrolyte ceramics F_{A}<18> and F_{B}<19> are fitted are not concave but tapered.

FIGS. 4 A to 4 D

FIGS. 4 A (=“FIG. 4 A”) to 4 D show further embodiments of preferred dividing walls W<16>.

The dividing wall W<16> shown in FIG. 4 A corresponds to the dividing wall W<16> shown in FIG. 2 A, except that it also comprises a frame element R<20>. This fully covers all the surfaces of the dividing wall W<16> except for O_{KK}<163> and O_{A/MK}<164>. The frame element R<20> is not in one-piece form together with the separating element T<17>.

FIG. 4 B (=“FIG. 4 B”) shows a further embodiment of a preferred dividing wall W<16>. This corresponds to the embodiment shown in FIG. 4 A, except that it comprises two frame elements R<20> that bound the upper and lower surfaces of the dividing wall W<16>.

FIG. 4 C (=“FIG. 4 C”) shows a further embodiment of a preferred dividing wall W<16>. The dividing wall W<16> shown in FIG. 4 C corresponds to the dividing wall W<16> shown in FIG. 2 B, except that it also comprises a frame element R<20>. This fully covers all the surfaces of the dividing wall W<16> except for O_{KK}<163> and O_{A/MK}<164>. The frame element R<20> is not in one-piece form together with the separating element T<17>.

FIG. 4 D (=“FIG. 4 D”) shows a further embodiment of a preferred dividing wall W<16>. This corresponds to the embodiment shown in FIG. 4 C, except that it comprises two frame elements R<20> that bound the upper and lower surfaces of the dividing wall W<16>.

FIGS. 5 A and 5 B

FIG. 5 A (=“FIG. 5 A”) shows an electrolysis cell E<1> in a preferred embodiment of the method according to the invention. This corresponds to the electrolysis cell shown in FIG. 1 A, with the difference that a dividing wall W<16> separates the interior I_{KK}<122> of the cathode chamber K_{K}<12> from the interior I_{KA}<112> of the anode chamber K_{A}<11>. The dividing wall is that shown in FIGS. 2 A and 2 B.

FIG. 5 B (=“FIG. 5 B”) shows an electrolysis cell E<1> in a preferred embodiment of the method according to the invention. This corresponds to the electrolysis cell shown in FIG. 1 A, with the difference that a dividing wall W<16> separates the interior I_{KK}<122> of the cathode chamber K_{K}<12> from the interior I_{KA}<112> of the anode chamber K_{A}<11>. The dividing wall W<16> is that shown in FIGS. 4 A to 4 D. The frame element R<20> forms part of the outer wall W_{A}<80>, such that the solid-state electrolyte ceramics encompassed by the dividing wall W<16> are protected from the pressure that would act thereon via the dividing wall W<16> if they were part of the dividing wall W<16>. In addition, the solid-state electrolyte ceramics are thus used entirely for separation of the interiors I_{KK}<122> and I_{KA}<112> within the electrolysis cell E<1>, since they are not partly covered by the outer wall.

FIGS. 6 A and 6 B

FIG. 6 A (=FIG. 6 A) shows the method according to the invention by way of an electrolysis cell E<1> corresponding to that shown in FIG. 1 B, with the difference that the connection V_{AM}<15> from the interior I_{KM}<132> of the middle chamber K_{M}<13> to the interior I_{KA}<112> of the anode chamber K_{A}<11> is formed by multiple perforations in the diffusion barrier D<14>. These perforations may be stamped subsequently into the diffusion barrier D<14> or may already have been present therein from the outset because of the production process for the diffusion barrier D<14> (for example in the case of textile fabrics such as filter cloths or metal weaves). In this embodiment, the totality of these perforations constitutes the connection V_{AM}<15> through which electrolyte can be directed from the interior I_{KM}<132> into the interior I_{KA}<112>.

FIG. 6 B (=“FIG. 6 B”) shows a further embodiment of the method according to the invention in the form of an electrolysis cell E<1>. This corresponds to the electrolysis cell E<1> shown in FIG. 1 B, with the difference that the connection V_{AM}<15> from the interior I_{KM}<132> of the middle chamber K_{M}<13> to the interior I_{KA}<112> of the anode chamber K_{A}<11> is formed by a gap that forms between the diffusion barrier D<14> and the outer wall W_{A}<80>. This gap can be set up in that an otherwise impervious diffusion barrier D<14> is disposed in the electrolysis cell E<1> such that it does not fully divide the interior I_{KM}<132> of the middle chamber K_{M}<13> from the interior I_{KA}<112> of the anode chamber K_{A}<11>, with a gap instead being retained as connection V_{AM}<15>.

FIGS. 7 A and 7 B

FIG. 7 A (=“FIG. 7 A”) shows a further embodiment of a preferred dividing wall W<16>. This comprises four NaSICON solid-state electrolyte ceramics F_{A}<18>, F_{B}<19>, F_{C}<28> and F_{D}<29> that are separated from one another by a separating element T<17> comprising two halves <171> and <172>. The dividing wall W<16> also comprises a frame element R<20> likewise consisting of two halves <201> and <202>.

The dividing wall W<16> consists of two parts that can be folded in, in which half <171> of the separating element T<17> is in one-piece form together with half <201> of the frame element R<20>, and half <172> of the separating element T<17> is in one-piece form together with half <202> of the frame element R<20>. These two parts may optionally be connected to one another via a hinge <50> and may be locked in place in the folded-in state via the lock <60>.

The four NaSICON solid-state electrolyte ceramics F_{A}<18>, F_{B}<19>, F_{C}<28> and F_{D}<29> are clamped between these halves, with use in each case of a ring that functions as seal Di<40> for sealing.

The left-hand side of FIG. 7 A shows the front view of the side S_{KK}<161> with the surface O_{KK}<163> of the dividing wall W<16>. The rings that function as seal Di<40> are shown by dotted outlines. The right-hand side of the figure shows the side view of the dividing wall W<16>.

FIG. 7 B (=“FIG. 7 B”) shows a further embodiment of a preferred dividing wall W<16>. This corresponds to the embodiment described in FIG. 7 A, except that it comprises nine NaSICON solid-state electrolyte ceramics F_{A}<18>, F_{B}<19>, F_{C}<28>, F_{D}<29>, F_{E}<30>, F_{F}<31>, F_{G}<32>, F_{H}<33>, F_{I}<34>.

FIG. 8

FIG. 8 (=FIG. 8) shows the comparison of the contents of BHET (“1”), 2-hydroxyethyl terephthalate (“MHET”; “2”) and terephthalate (“TS”; “3”) in the depolymerization with sodium glycolate obtained by the method according to the invention, and sodium glycolate obtained by conventional methods.

The bars with the close hatching “///////” show the respective content of BHET, MHET and TS in the reactor output in the depolymerization of PET according to Inventive Example E1 in which the sodium glycolate used for the depolymerization was obtained by electrolysis.

The black bars show the respective content of BHET, MHET and TS in the reactor output in the depolymerization of PET according to Comparative Example V1 in which merely glycol was used in the depolymerization.

The bars with the bold hatching “/////” show the respective content of BHET, MHET and TS in the reactor output in the depolymerization of PET according to Comparative Example V2 in which the sodium glycolate used for the depolymerization was obtained by mixing NaOH and glycol in the reactor.

### DETAILED DESCRIPTION OF THE INVENTION

It has now been found that, surprisingly, the glycolysis of PET proceeds particularly efficiently when alkali metal glycolate, especially sodium glycolate or potassium glycolate, that has been obtained electrolytically is used. It has been observed that, in the method according to the invention, by comparison with the prior art methods in which glycolate that has been obtained by dissolution of the corresponding alkali metal hydroxides in glycol is used, a higher proportion of BHET is obtained in the cleavage product.

1. Step (a): Electrolysis to Obtain the Solution L₁ Comprising Glycol and M_{A} Glycolate

According to the invention, the solution L₁ comprising glycol and M_{A} glycolate which is used in the method according to the invention is obtained electrolytically in an electrolysis cell E<1>.

“Glycol” in the context of the invention is understood to mean ethylene-1,2-diol having the chemical formula HO—CH₂—CH₂—OH (CAS No. 107-21-1).

“M_{A} glycolate” in the context of the invention is understood to mean the salt of glycol with M_{A}. The term “M_{A} glycolate” encompasses at least one of M_{A}O—CH₂—CH₂—OH and M_{A}O—CH₂—CH₂—OM_{A}, preferably at least M_{A}O—CH₂—CH₂—OH, most preferably M_{A}O—CH₂—CH₂—OH and M_{A}O—CH₂—CH₂—OM_{A}.

M_{A} is an alkali metal cation, especially selected from lithium, sodium, potassium, and preferably from sodium, potassium. Most preferably, the alkali metal cation is sodium.

### 1.1 Electrolysis Cell E

The solution L₁<21> of M_{A} glycolate in glycol which is used in step (b) of the method according to the invention is prepared in an electrolysis cell E in step (a) of the method according to the invention.

The electrolysis cell E comprises at least one anode chamber K_{A} and at least one cathode chamber K_{K}, and optionally at least one interposed middle chamber K_{M}. This also includes electrolysis cells E having more than one anode chamber K_{A} and/or cathode chamber K_{K} and/or middle chamber K_{M}. Such electrolysis cells in which these chambers are joined to one another in the form of modules are described, for example, in DD 258 143 A3 and US 2006/0226022 A1.

The electrolysis cell E, in a preferred embodiment, comprises an anode chamber K_{A} and a cathode chamber K_{K}, and optionally an interposed middle chamber K_{M}.

The electrolysis cell E typically has an outer wall W_{A}. The outer wall W_{A} is especially made from a material selected from the group consisting of steel, preferably rubberized steel, plastic, especially from Telene® (thermoset polydicyclopentadiene), PVC (polyvinylchloride), PVC-C (post-chlorinated polyvinylchloride), PVDF (polyvinylidenefluoride). W_{A} may especially be perforated for inlets and outlets. Within W_{A} are then the at least one anode chamber K_{A}, the at least one cathode chamber K_{K} and, In the embodiments in which the electrolysis cell E comprises one, the at least one interposed middle chamber K_{M}.

### 1.1.1 Cathode Chamber K_{K}

The at least one cathode chamber K_{K} has at least one inlet Z_{KK}, at least one outlet A_{KK}, and an interior I_{KK} comprising a cathodic electrode E_{K}.

The interior I_{KA} of the anode chamber K_{A} is divided from the interior I_{KK} of the cathode chamber K_{K} by a dividing wall W if the electrolysis cell E does not comprise a middle chamber K_{M}. The interior I_{KK} of the cathode chamber K_{K} is divided from the interior I_{KM} of the middle chamber K_{M} by a dividing wall W if the electrolysis cell E comprises at least one middle chamber K_{M}. The dividing wall W and the arrangement thereof in the electrolysis cell E is described further down (section 1.1.4).

### 1.1.1.1 Cathodic Electrode E_{K}

The cathode chamber K_{K} comprises an interior I_{KK} which in turn comprises a cathodic electrode E_{K}. A useful cathodic electrode E_{K} of this kind is any electrode familiar to the person skilled in the art that is stable under the conditions of step (a) of the method according to the invention. These are described, in particular, in WO 2014/008410 A1, paragraph or DE 10360758 A1, paragraph [030]. This electrode Ex may be selected from the group consisting of mesh wool, three-dimensional matrix structure and “balls”. The cathodic electrode Ex especially comprises a material selected from the group consisting of steel, nickel, copper, platinum, platinized metals, palladium, carbon-supported palladium, titanium, more preferably selected from the group consisting of steel, nickel. Ex preferably comprises steel, even more preferably VA steel (=stainless steel).

In the embodiments of the electrolysis cell E in which it comprises a middle chamber K_{M}, this is located between the anode chamber K_{A} and the cathode chamber K_{K}.

1.1.1.2 Inlet Z_{KK} and Outlet A_{KK}

The cathode chamber K_{K} also comprises at least one inlet Z_{KK} and at least one outlet A_{KK}. This enables the addition of liquid, for example the solution L₂, to the interior I_{KK} of the cathode chamber K_{K}, and removal of liquid present therein, for example the solution L₁. The inlet Z_{KK} and the outlet A_{KK} are attached here to the cathode chamber K_{K} in such a way that the liquid comes into contact with the cathodic electrode Ex as it flows through the interior I_{KK} of the cathode chamber K_{K}. This is a prerequisite in order for the solution L₁ to be obtained at the outlet A_{KK} in the performance of step (a) of the method according to the invention when the solution L₂ of glycol, optionally also comprising an M_{A} glycolate, is directed through the interior I_{KK} of the cathode chamber K_{K}.

The inlet Z_{KK} and the outlet A_{KK} may be attached to the electrolysis cell E by methods known to the person skilled in the art, for example by means of holes in the outer wall and corresponding connections (valves) that simplify the introduction and discharge of liquid.

### 1.1.2 Anode Chamber K_{A}

The at least one anode chamber K_{A} has at least one inlet Z_{KA}, at least one outlet A_{KA}, and an interior I_{KA} comprising an anodic electrode E_{A}.

The interior I_{KA} of the anode chamber K_{A}, if the electrolysis cell E comprises a middle chamber K_{M}, is divided from the interior I_{KM} of the middle chamber K_{M} by a diffusion barrier D.

If electrolysis cell E does not comprise a middle chamber K_{M}, the interior I_{KA} of the anode chamber K is divided from the interior I_{KK} of the cathode chamber K_{K} by the dividing wall W.

### 1.1.2.1 Anodic Electrode E_{A}

The anode chamber K_{A} comprises an interior I_{KA} which in turn comprises an anodic electrode E_{A}. A useful anodic electrode E_{A} of this kind is any electrode familiar to the person skilled in the art that is stable under the conditions of step (a) of the method according to the invention. These are described, in particular, in WO 2014/008410 A1, paragraph or DE 10360758 A1, paragraph [031]. This electrode E_{A} may consist of one layer or consist of multiple planar layers parallel to one another that may each be perforated or expanded. The anodic electrode E_{A} especially comprises a material selected from the group consisting of ruthenium oxide, iridium oxide, nickel, cobalt, nickel tungstate, nickel titanate, noble metals such as, in particular, platinum, supported on a support such as titanium or Kovar® (an iron/nickel/cobalt alloy in which the individual components are preferably as follows: 54% by mass of iron, 29% by mass of nickel, 17% by mass of cobalt). Further possible anode materials are especially stainless steel, lead, graphite, tungsten carbide, titanium diboride. Preferably, the anodic electrode E_{A} comprises a titanium anode coated with ruthenium oxide/iridium oxide (RuO₂+IrO₂/Ti).

1.1.2.2 Inlet Z_{KA} and Outlet A_{KA}

The anode chamber K_{K} also comprises an inlet Z_{KA} and an outlet A_{KA}. This enables the addition of liquid, for example the solution L₃, to the interior I_{KA} of the cathode chamber K_{A}, and removal of liquid present therein, for example the solution L₄. The inlet Z_{KA} and the outlet A_{KA} are attached here to the anode chamber K_{A} in such a way that the liquid comes into contact with the anodic electrode E_{A} as it flows through the interior I_{KA} of the anode chamber K_{A}. This is a prerequisite in order for the solution L₄ to be obtained at the outlet A_{KA} in the performance of step (a) of the process of the invention when the solution L₃ of a salt S is directed through the interior I_{KA} of the anode chamber K_{A}.

The inlet Z_{KA} and the outlet A_{KA} may be attached to the electrolysis cell E by methods known to the person skilled in the art, for example by means of holes in the outer wall and corresponding connections (valves) that simplify the introduction and discharge of liquid. The inlet Z_{KA}, in particular embodiments in which the electrolysis cell E comprises a middle chamber K_{M}, may also be within the electrolysis cell, for example in the form of a perforation in the diffusion barrier D.

### 1.1.3 Optional Middle Chamber K_{M}

The electrolysis cell E used in step (a) of the method according to the invention optionally has at least one middle chamber K_{M}. The optional middle chamber K_{M} lies between cathode chamber K_{K} and anode chamber K_{A}. It comprises at least one inlet Z_{KM}, at least one outlet A_{KM} and an interior I_{KM}.

The interior I_{KA} of the anode chamber K_{A}, if the electrolysis cell E comprises a middle chamber K_{M}, is divided from the interior I_{KM} of the middle chamber K_{M} by a diffusion barrier D. A_{KM} in that case is also connected to the inlet Z_{KA} by a connection V_{AM}, such that liquid can be directed from I_{KM} into I_{KA} through the connection V_{AM}.

### 1.1.3.1 Diffusion Barrier D

The interior I_{KM} of the optional middle chamber K_{M} is divided from the interior I_{KA} of the anode chamber K_{A} by a diffusion barrier D and divided from the interior I_{KK} of the cathode chamber K_{K} by the dividing wall W.

The material used for the diffusion barrier D may be any which is stable under the conditions of step (a) of the method according to the invention and prevents or slows the transfer of protons from the liquid present in the interior I_{KA} of the anode chamber K_{A} to the interior I_{KM} of the optional middle chamber K_{M}.

The diffusion barrier D used is especially a non-ion-specific dividing wall or a membrane permeable to specific ions. The diffusion barrier D is preferably a non-ion-specific dividing wall.

The material of the non-ion-specific dividing wall is especially selected from the group consisting of fabric, which is especially textile fabric or metal weave, glass, which is especially sintered glass or glass frits, ceramic, especially ceramic frits, membrane diaphragms, and is more preferably a textile fabric or metal weave, especially preferably a textile fabric. The textile fabric preferably comprises plastic, more preferably a plastic selected from PVC, PVC-C, polyvinylether (“PVE”), polytetrafluoroethylene (“PTFE”).

If the diffusion barrier D is a “membrane permeable to specific ions”, what this means in accordance with the invention is that the respective membrane promotes the diffusion therethrough of particular ions over other ions. More particularly, what this means is membranes that promote the diffusion therethrough of ions of a particular charge type over ions of the opposite charge. Even more preferably, membranes permeable to specific ions also promote the diffusion therethrough of particular ions of one charge type over other ions of the same charge type.

If the diffusion barrier D is a “membrane permeable to specific ions”, the diffusion barrier D is especially an anion-conducting membrane or a cation-conducting membrane.

According to the invention, anion-conducting membranes are those that selectively conduct anions, preferably selectively conduct particular anions. In other words, they promote the diffusion therethrough of anions over that of cations, especially over protons; even more preferably, they additionally promote the diffusion therethrough of particular anions over the diffusion therethrough of other anions.

According to the invention, cation-conducting membranes are those that selectively conduct cations, preferably selectively conduct particular cations. In other words, they promote the diffusion therethrough of cations over that of anions; even more preferably, they additionally promote the diffusion therethrough of particular cations over the diffusion therethrough of other cations, more preferably still that of cations that are not protons, more preferably sodium cations, over protons.

What is meant more particularly by “promote the diffusion of particular ions X over the diffusion of other ions Y” is that the coefficient of diffusion (unit: m²/s) of ion type X at a given temperature for the membrane in question is higher by a factor of 10, preferably 100, preferably 1000, than the coefficient of diffusion of ion type Y for the membrane in question.

If the diffusion barrier D is a “membrane permeable to specific ions”, it is preferably an anion-conducting membrane, since this particularly efficiently prevents the diffusion of protons from the anode chamber K_{A} into the middle chamber K_{M}.

The anion-conducting membrane used is especially one selective for the anions encompassed by the salt S. Such membranes are known to and can be used by the person skilled in the art. According to the invention, the salt S comprises M_{A} as cation.

The salt S is preferably a halide, sulfate, sulfite, nitrate, hydrogen carbonate or carbonate of M_{A}, even more preferably a halide.

Halides are fluorides, chlorides, bromides, iodides. The most preferred halide is chloride.

The anion-conducting membrane used is preferably one selective for halides, preferably chloride.

Anion-conducting membranes are described, for example, by M. A. Hickner, A. M. Herring, E. B. Coughlin, Journal of Polymer Science, Part B: Polymer Physics 2013, 51, 1727-1735, by C. G. Arges, V. Ramani, P. N. Pintauro, Electrochemical Society Interface 2010, 19, 31-35, in WO 2007/048712 A2, and on page 181 of the textbook by Volkmar M. Schmidt, Elektrochemische Verfahrenstechnik: Grundlagen, Reaktionstechnik, Prozessoptimierung [Electrochemical Engineering: Fundamentals, Reaction Technology, Process Optimization], 1st edition (8 Oct. 2003).

Even more preferably, anion-conducting membranes used are accordingly organic polymers that are especially selected from polyethylene, polybenzimidazoles, polyether ketones, polystyrene, polypropylene and fluorinated membranes such as polyperfluoroethylene, preferably polystyrene, where these have covalently bonded functional groups selected from —NH₃⁺, —NRH₂⁺, —NR₃⁺, ═NR⁺; —PR₃⁺, where R is alkyl groups having preferably 1 to 20 carbon atoms, or other cationic groups. They preferably have covalently bonded functional groups selected from —NH₃⁺, —NRH₂⁺ and —NR₃⁺, more preferably selected from —NH₃⁺ and —NR₃⁺, even more preferably —NR₃⁺.

When the diffusion barrier D is a cation-conducting membrane, it is especially a membrane selective for M_{A}, i.e. the cation encompassed by the salt S. Even more preferably, the diffusion barrier D is an alkali metal cation-conducting membrane, even more preferably a potassium and/or sodium ion-conducting membrane, most preferably a sodium ion-conducting membrane.

Cation-conducting membranes are described, for example, on page 181 of the textbook by Volkmar M. Schmidt, Elektrochemische Verfahrenstechnik: Grundlagen, Reaktionstechnik, Prozessoptimierung, 1st edition (8 Oct. 2003).

Even more preferably, cation-conducting membranes used are accordingly organic polymers that are especially selected from polyethylene, polybenzimidazoles, polyether ketones, polystyrene, polypropylene and fluorinated membranes such as polyperfluoroethylene, preferably polystyrene and polyperfluoroethylene, where these bear covalently bonded functional groups selected from —SO₃, —COO, —PO₃²⁻ and —PO₂H⁻, preferably—SO₃⁻ (described in DE 10 2010 062 804 A1, U.S. Pat. No. 4,831,146).

This may be, for example, a sulfonated polyperfluoroethylene (Nafion® with CAS number: 31175-20-9). These are known to the person skilled in the art, for example from WO 2008/076327 A1, paragraph [058], US 2010/0044242 A1, paragraph [0042] or US 2016/0204459 A1, and are commercially available under the Nation®, Aciplex @ F, Flemion®, Neosepta®, Ultrex®, PC-SK® trade names. Neosepta@ membranes are described, for example, by S. A. Mareev, D. Yu. Butylskii, N. D. Pismenskaya, C. Larchet, L. Dammak, V. V. Nikonenko, Journal of Membrane Science 2018, 563, 768-776.

If a cation-conducting membrane is used as diffusion barrier D, this may, for example, be a polymer functionalized with sulfonic acid groups, especially of the formula P_{NAFION} below, where n and m may independently be an integer from 1 to 106, preferably an integer from 10 to 105, more preferably an integer from 102 to 104.

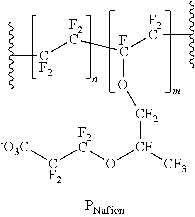

1.1.3.2 Inlet Z_{KM} and Outlet A_{KM}

The optional middle chamber K_{M} also comprises an inlet Z_{KM} and an outlet A_{KM}. This enables the addition of liquid, for example the solution L₃, to the interior I_{KM} of the middle chamber K_{M}, and the transfer of liquid present therein, for example the solution L₃, to the interior I_{KA} of the anode chamber K_{A}.

The inlet Z_{KM} and the outlet A_{KM} may be attached to the electrolysis cell E by methods known to the person skilled in the art, for example by means of holes in the outer wall and corresponding connections (valves) that simplify the introduction and discharge of liquid. The outlet A_{KM} may also be within the electrolysis cell, for example in the form of a perforation in the diffusion barrier D.

### 1.1.3.3 Connection V_{AM}

In the electrolysis cell E used in step (a) of the method according to the invention, the outlet A_{KM} is connected to the inlet Z_{KA} by a connection V_{AM} in such a way that liquid can be directed from I_{KM} into I_{KA} through the connection V_{AM}.

The connection V_{AM} may be formed within the electrolysis cell E and/or outside the electrolysis cell E, and is preferably formed within the electrolysis cell.

1) If the connection V_{AM} is formed within the electrolysis cell E, it is preferably formed by at least one perforation in the diffusion barrier D. This embodiment is preferred especially when the diffusion barrier D used is a non-ion-specific dividing wall, especially a metal weave or textile fabric. This functions as a diffusion barrier D and, on account of the weave properties, has perforations and gaps from the outset that function as connection V_{AM}.

2) The embodiment described hereinafter is preferred especially when the diffusion barrier D used is a membrane permeable to specific ions: In this embodiment, the connection V_{AM} is formed outside the electrolysis cell E, preferably formed by a connection of A_{KM} and Z_{KA} that runs outside the electrolysis cell E, especially in that an outlet A_{KM} through the outer wall W_{A} is formed from the interior I_{KM} of the middle chamber K_{M}, preferably at the base of the middle chamber K_{M}, the inlet Z_{KM} more preferably being at the top end of the middle chamber K_{M}, and an inlet Z_{KA} through the outer wall W_{A} is formed in the interior I_{KA} of the anode chamber K_{A}, preferably at the base of the anode chamber K_{A}, and these are connected by a conduit, for example a pipe or a hose, preferably comprising a material selected from rubber and plastic. The outlet A_{KA} is then more preferably at the top end of the anode chamber K_{A}.

“Outlet A_{KM} at the base of the middle chamber K_{M}” means that the outlet A_{KM} is attached to the electrolysis cell E in such a way that the solution L₃ leaves the middle chamber K_{M} in the direction of gravity.

“Inlet Z_{KA} at the base of the anode chamber K_{A}” means that the inlet Z_{KA} is attached to the electrolysis cell E in such a way that the solution L₃ enters the anode chamber K_{A} counter to gravity.

“Inlet Z_{KM} at the top end of the middle chamber K_{M}” means that the inlet Z_{KM} is attached to the electrolysis cell E in such a way that the solution L₃ enters the middle chamber K_{M} in the direction of gravity.

“Outlet A_{KA} at the top end of the anode chamber K_{A}” means that the outlet A_{KA} is mounted on the electrolysis cell E in such a way that the solution La leaves the anode chamber K_{A} counter to gravity.

This embodiment is particularly advantageous and therefore preferred when the outlet A_{KM} is formed by the outer wall W_{A} at the base of the middle chamber K_{M}, and the inlet Z_{KA} by the outer wall W_{A} at the base of the anode chamber K_{A}. This arrangement makes it possible in a particularly simple manner to remove gases formed in the anode chamber K_{A} from the anode chamber K_{A} with L₄, in order to separate them further. FIG. 1 B shows such an embodiment.

When the connection V_{AM} is formed outside the electrolysis cell E, Z_{KM} and A_{KM} are especially arranged at opposite ends of the outer wall W_{A} of the middle chamber K_{M} (i.e., for example, Z_{KM} at the base and A_{KM} at the top end of the electrolysis cell E or vice versa) and Z_{KA} and A_{KA} are arranged at opposite ends of the outer wall W_{A} of the anode chamber K_{A} (i.e. Z_{KA} at the base and A_{KA} at the top end of the electrolysis cell E or vice versa), as shown more particularly in FIG. 1 B. By virtue of this geometry, L₃ must flow through the two chambers K_{M} and K_{A}. It is possible here for Z_{KA} and Z_{KM} to be formed on the same side of the electrolysis cell E, in which case A_{KM} and A_{KA} are automatically also formed on the same side of the electrolysis cell E. Alternatively, Z_{KA} and Z_{KM} may, as in the embodiment shown in FIG. 1 B, be formed on opposite sides of the electrolysis cell E, in which case A_{KM} and A_{KA} are then automatically also formed on opposite sides of the electrolysis cell E.

3) When the connection V_{AM} is formed within the electrolysis cell E, this may especially be ensured in that one side (“side A”) of the electrolysis cell E, which is the top end or the base of the electrolysis cell E, preferably the top end as shown in FIG. 6 B, comprises the inlet Z_{KM} and the outlet A_{KA}, and the diffusion barrier D extends proceeding from this side (“side A”) into the electrolysis cell E, but does not quite reach up to the side (“side B”) of the electrolysis cell E opposite side A, which is then the base or the top end of the electrolysis cell E, and at the same time covers 50% or more of the height of the three-chamber cell E, preferably 60% to 99% of the height of the three-chamber cell E, more preferably 70% to 95% of the height of the three-chamber cell E, even more preferably 80% to 90% of the height of the three-chamber cell E, more preferably still 85% of the height of the three-chamber cell E. Because the diffusion barrier D does not touch side B of the three-chamber cell E, a gap thus arises between diffusion barrier D and the outer wall W_{A} of side B of the three-chamber cell E. In that case, the gap is the connection V_{AM}. By virtue of this geometry, L₃ must flow completely through the two chambers K_{M} and K_{A}.

These embodiments best ensure that the aqueous salt solution L₃ flows past the acid-sensitive solid-state electrolyte before it comes into contact with the anodic electrode E_{A}, which results in the formation of acids.

According to the invention, “base of the electrolysis cell E” is the side of the electrolysis cell E through which a solution (e.g. L₃ in the case of A_{KM} in FIG. 1 B) exits from the electrolysis cell E in the same direction as gravity, or the side of the electrolysis cell E through which a solution (e.g. L₂ in the case of Z_{KK} in FIGS. 1 A, 1 B, 6 A and 6 B, and L₃ in the case of Z_{KA} in FIGS. 1 A and 1 B) is supplied to the electrolysis cell E counter to gravity.

According to the invention, “top end of the electrolysis cell E” is the side of the electrolysis cell E through which a solution (e.g. L₄ in the case of A_{KA} and L₁ in the case of A_{KK} in FIGS. 1 A, 1 B, 6 A and 6 B) exits from the electrolysis cell E counter to gravity, or the side of the electrolysis cell E through which a solution (e.g. L₃ in the case of Z_{KM} in FIGS. 1 B, 6 A and 6 B) is supplied to the electrolysis cell E in the same direction as gravity.

### 1.1.3.4 Further Embodiments of the Middle Chamber K_{M}

In a preferred embodiment of the electrolysis cell E, the interior I_{KM} also comprises at least one additional feature selected from:
1) internals set up such that they lead to turbulence in the electrolyte L₃;
2) a stirring apparatus;
3) an additional introduction of an inert gas (e.g. nitrogen or noble gas) via an additional inlet at the bottom end of the middle chamber and an additional outlet at the top end of the middle chamber. By means of this additional outlet, it is also possible to remove any gases formed, for example CO₂, when the salt S is a carbonate or hydrogen carbonate, from I_{KM}.

By virtue of these additional preferred embodiments 1), 2) and 3), vortexes and turbulence form in the electrolyte L₃ as it flows through I_{KM}. This additionally hinders the formation of a pH gradient in the middle chamber, and hence prevents damage to the ASC as a result of too low a pH. This increases the lifetime of the ASC.

### 1.1.4 Dividing Wall W

The electrolysis cell E used in step (a) of the method according to the invention comprises a dividing wall W. The dividing wall W comprises at least one alkali metal cation-conducting solid-state electrolyte ceramic F_{A}. In a preferred embodiment, the dividing wall W consists of an alkali metal cation-conducting solid-state electrolyte ceramic F_{A}.

In an alternative preferred embodiment of the present invention, the dividing wall W comprises at least two alkali metal cation-conducting solid-state electrolyte ceramics (“alkali metal cation-conducting solid-state electrolyte ceramic” is abbreviated hereinafter as “ASC”) F_{A} and F_{B}, optionally separated from one another by a separating element T.

The dividing wall W has two sides S_{KK} and S_{A/MK} that are opposite one another, meaning that side S_{A/MK} is opposite side S_{KK} (and vice versa). The two sides S_{KK} and S_{A/MK} especially comprise planes that are essentially parallel to one another.

The geometry of the dividing wall W is otherwise subject to no further restriction, and may be matched in particular to the cross section of the electrolysis cell E in which it is used. For example, it may have the geometry of a cuboid and hence have a rectangular cross section, or the geometry of a frustocone or cylinder and accordingly a circular cross section.

Optionally, the dividing wall W may also have the geometry of a cuboid with rounded corners or bulges which may in turn have holes. The dividing wall W then has bulges (“rabbit's ears”) by which the dividing wall W can be fixed to electrolysis cells, or else frame parts of the dividing wall W can be fixed to one another.

The side S_{KK} of the dividing wall W has the surface O_{KK}, and the side S_{A/MK} of the dividing wall W has the surface O_{A/MK}.

What is meant by the feature “dividing wall” is that the dividing wall W is liquid-tight. Thus, there exist no gaps through which aqueous solution, alcoholic solution, alcohol or water could flow from the S_{KK} side to the S_{A/MK} Side or vice versa. In the cases in which the dividing wall W comprises at least two alkali metal cation-conducting solid-state electrolyte ceramics F_{A} and F_{B} and optionally a separating element T, this means that F_{A} and F_{B} and the at least one separating element T, if present, adjoin one another in a gapless manner.

The dividing wall W usable in the electrolysis cell E in step (a) of the method according to the invention also encompasses embodiments in which the dividing wall W comprises more than two ASCs, for example four or nine or twelve ASCs, where the ASCs either directly adjoin one another or are separated from one another by a separating element T.

When the ASCs directly adjoin one another, however, this requires an exact fit of the respectively adjoining ASCs in order to avoid the formation of a gap between them, through which aqueous liquid or water or glycol or glycolic solution could flow from the S_{KK} side to the S_{A/MK} side. It is therefore advantageous and preferable that, when the dividing wall W comprises more than one ASC, all ASCs encompassed by the dividing wall W are separated from one another by at least one separating element T in the dividing wall W, meaning that no ASC directly adjoins any other ASC, i.e. without a separating element T in between.

The dividing wall W is further characterized in that the ASC F_{A} encompassed by the dividing wall W is directly contactable both via the surface O_{KK} and via the surface O_{A/MK}. In the embodiment in which the dividing wall W comprises at least two ASCs F_{A}, F_{B}, it is preferable that all ASCs encompassed by the dividing wall W are directly contactable both via the surface Ok and via the surface O_{A/MK}.

What is meant by “directly contactable” with regard to the ASCs encompassed by the dividing wall W is that at least part of the surfaces O_{KK} and O_{A/MK} is formed by the surface of the ASCs encompassed by the dividing wall W, meaning that the ASCs encompassed by the dividing wall W are directly accessible at the two surfaces O_{KK} and O_{A/MK}, such that they can be wetted at the two surfaces O_{KK} and O_{A/MK}, for example, with aqueous solution, glycolic solution, glycol or water.

What this means for the arrangement of the ASCs in the dividing wall W is that, for each ASC encompassed by the dividing wall W, there is a route from the surface O_{KK} on the S_{KK} side to the surface O_{A/MK} on the S_{A/MK} side that leads completely through the respective ASC.

When the dividing wall W has at least one separating element T, the at least one separating element T is also typically directly contactable both via at least part of the surface O_{KK} and via at least part of the surface O_{A/MK}.

What is meant by “directly contactable” with regard to the at least one separating element T optionally encompassed by the dividing wall W is that part of the surfaces O_{KK} and O_{A/MK} is formed by the surface of the separating element T, meaning that the separating element T is directly accessible at the two surfaces O_{KK} and O_{A/MK}, such that the separating element T can be wetted at the two surfaces O_{KK} and O_{A/MK}, for example, with aqueous solution, alcoholic solution, alcohol or water.

What this means more particularly in respect of the arrangement of the optional separating element T in the dividing wall W is that, for the separating element T optionally encompassed by the dividing wall W, there is a route from the surface OK on the S_{KK} side to the surface O_{A/MK} on the S_{A/MK} side that leads through the separating element T, and optionally through a seal Di, but not through an ASC.

In a preferred embodiment of the dividing wall W, at least 50%, more preferably at least 60%, even more preferably at least 70%, even more preferably at least 85%, of the surface O_{A/MK} is formed by the ASCs encompassed by the dividing wall W.

In a preferred embodiment of the dividing wall W, at least 50%, more preferably at least 60%, even more preferably at least 70%, even more preferably at least 85%, of the surface O_{KK} is formed by the ASCs encompassed by the dividing wall W.

In the embodiment in which the dividing wall W has more than one ASC, in particular, 50% to 99%, more preferably at least 60% to 96%, even more preferably 70% to 92%, even more preferably 85% to 90%, of the surface O_{KK} is formed by the ASCs encompassed by the dividing wall W, with the rest of the surface O_{KK} even more preferably being formed by the separating element T and optionally the frame element R. At the same time, in the embodiment in which the dividing wall W has more than one ASC, in particular, 50% to 99%, more preferably at least 60% to 96%, even more preferably 70% to 92%, even more preferably 85% to 90%, of the surface O_{A/MK} is formed by the ASCs encompassed by the dividing wall W, with the rest of the surface O_{A/MK} even more preferably being formed by the separating element T and optionally the frame element R.

In the preferred embodiment, the dividing wall W<16> comprises an alkali metal cation-conducting solid-state electrolyte ceramic F_{A} and optionally a frame element R. Even more preferably, the dividing wall W<16> consists of an alkali metal cation-conducting solid-state electrolyte ceramic F_{A}.

In another preferred embodiment, the dividing wall W comprises at least four ASCs F_{A}, F_{B}, F_{C} and F_{D}, and even more preferably comprises exactly four ASCs F_{A}, F_{B}, F_{C} and F_{D}.

In a further preferred embodiment, the dividing wall W comprises at least nine ASCs F_{A}, F_{B}, F_{C}, F_{D}, F_{E}, F_{F}, F_{G}, F_{H} and F_{I}, and even more preferably comprises exactly nine ASCs F_{A}, F_{B}, F_{C}, F_{D}, F_{E}, F_{F}, F_{G}, F_{H} and F_{I}.

In a further preferred embodiment, the dividing wall W comprises at least twelve ASCs F_{A}, F_{B}, F_{C}, F_{D}, F_{E}, F_{F}, F_{G}, F_{H}, F_{I}, F_{J}, F_{K} and F_{L}, and even more preferably comprises exactly twelve ASCs F_{A}, F_{B}, F_{C}, F_{D}, F_{E}, F_{F}, F_{G}, F_{H}, F_{I}, F_{J}, F_{K} and F_{L}.

The arrangement of at least two ASCs alongside one another in the dividing wall W results in an advantage over the arrangement of just one ASC, namely a further direction of spread for the ASCs in the event of the fluctuations in temperature that arise in the operation of the electrolysis cell. NaSICON sheets that function as dividing walls are bounded in electrolysis cells by the outer walls of the electrolysis cell or by solid plastic frames. It is not possible in this way to dissipate the mechanical stresses that occur in the event of expansion within the NaSICON, which can lead to fracture of the ceramic.

By contrast, the individual ASCs within the dividing wall W preferably adjoin the separating element T, which leads to two advantageous effects, both of which increase the long-term stability of the ASC:
each ASC has a further available degree of freedom, i.e. a dimension in which it can expand. As well as expansion in z direction (i.e. beyond the thickness of the ceramic sheet at right angles to the plane of the dividing wall W), expansion in x and/or y direction is now also possible, i.e. in horizontal and vertical direction within the plane of the dividing wall W. This direction of expansion does not exist, or is at least greatly restricted, when the ASCs, for example as a solid sheet, span the cross section of the electrolysis cell and adjoin the solid wall of the electrolysis cell.
compared to a dividing wall of equal size consisting solely of one ASC, the division into multiple small ASCs has the effect that the stresses that occur within the smaller ASCs are also smaller in absolute terms, can be dissipated more rapidly and hence cannot as quickly build up to a stress that leads to the fracture of the ASC.

As a result, the tendency to fracture is distinctly reduced for the “divided” ASCs in the dividing wall W compared to the use of one sheet.

### 1.1.4.1 Alkali Metal Cation-Conducting Solid-State Electrolyte Ceramic “ASC”

A useful alkali metal cation-conducting solid-state electrolyte ceramic F_{A}, F_{B} etc. encompassed by the dividing wall W is any solid-state electrolyte through which cations, especially alkali metal cations, even more preferably sodium cations, can be transported from the S_{A/MK} side to the S_{KK} side. Such solid-state electrolytes are known to the person skilled in the art and are described, for example, in DE 10 2015 013 155 A1, in WO 2012/048032 A2, paragraphs [0035], [0039], [0040], in US 2010/0044242 A1, paragraphs [0040], [0041], in DE 10360758 A1, paragraphs [014] to [025]. They are sold commercially under the NaSICON, LiSICON, KSICON name. A sodium ion-conducting solid-state electrolyte is preferred, and this even more preferably has an NaSICON structure. NaSICON structures usable in accordance with the invention are also described, for example, by N. Anantharamulu, K. Koteswara Rao, G. Rambabu, B. Vijaya Kumar, Velchuri Radha, M. Vithal, J Mater Sci 2011, 46, 2821-2837.

In a preferred embodiment of the dividing wall W, the alkali metal cation-conducting solid-state electrolyte ceramics encompassed by the dividing wall W, and especially the ASCs F_{A}, independently have an NaSICON structure of the formula M^{I}_{1+2w+x-y+z} M^{II}_{w}M^{III}ₓZr^{IV}_{2-w-x-y}M^{V}_{y}(SiO₄)_{z}(PO₄)_{3-z}.

M^{I} is here selected from Na⁺, Li⁺, preferably Na⁺.

M^{II} is here a divalent metal cation, preferably selected from Mg²⁺, Ca²⁺, Sr²⁺, Ba²⁺, Co²⁺, Ni²⁺, more preferably selected from Co²⁺, Ni²⁺.

M^{III} is here a trivalent metal cation, preferably selected from Al³⁺, Ga³⁺, Sc³⁺, La³⁺, Y³⁺, Gd³⁺, Sm³⁺, Lu³⁺, Fe³⁺, Cr³⁺, more preferably selected from Sc³⁺, La³⁺, Y³⁺, Gd³⁺, Sm³⁺, especially preferably selected from Sc³⁺, Y³⁺, La³⁺.

M^{V} is here a pentavalent metal cation, preferably selected from V⁵⁺, Nb⁵⁺, Ta⁵⁺.

The Roman indices I, II, III, IV, V indicate the oxidation numbers in which the respective metal cations exist.

w, x, y, z are real numbers, where 0≤x<2, 0≤y<2, 0≤w<2, 0≤z<3, and where w, x, y, z are chosen such that 1+2w+x−y+z≥0 and 2−w−x−y≥0.

Even more preferably in accordance with the invention, the NaSICON structure has a structure of the formula Na₍₁₊ᵥ₎Zr₂SiᵥP₍₃₋ᵥ₎O₁₂ where v is a real number for which 0≤v≤3. Most preferably, v=2.4

In a preferred embodiment of the dividing wall W in which it comprises at least two ASCs F_{A}, F_{B}, all ASCs encompassed by the dividing wall W have the same structure.

### 1.1.4.2 Separating Element T

In the embodiments of the inventive dividing wall W in which it comprises at least two ASCs F_{A}, F_{B}, the dividing wall W preferably comprises a separating element T. In that case, according to the invention, the separating element T separates at least two alkali metal cation-conducting solid-state electrolyte ceramics F_{A} and F_{B} encompassed by the dividing wall W, meaning that it is disposed between at least two alkali metal cation-conducting solid-state electrolyte ceramics F_{A} and F_{B} encompassed by the dividing wall W.

A suitable separating element T which is preferably encompassed by the dividing wall W is any body by means of which the respective ASCs can be arranged separately from one another. The ASCs here gaplessly adjoin the separating element T in order not to impair the function of the dividing wall, which, in the electrolysis cell E, is to divide the cathode chamber in a liquid-tight manner from the adjacent middle chamber or anode chamber.

The shape of the separating element T can be chosen by the person skilled in the art depending on the number of ASCs encompassed by the dividing wall W in the preferred embodiment.

If the dividing wall W comprises two or three ASCs, for example, these may each be separated by a land disposed between the ASCs as separating element T.

If the dividing wall W comprises four or more ASCs, these may be separated by a separating element T in the form of a cross or grid.

In the embodiments of the inventive dividing wall W in which it comprises at least two ASCs F_{A}, F_{B}, it is particularly preferable that the dividing wall W comprises at least four ASCs, and even more preferable that the separating element T in that case is in the form of a cross or grid, since it is then ensured that all three dimensions are fully available to the ASCs for thermal expansion/shrinkage.

The separating element T here may consist of one piece. In that case, the ASC is secured gaplessly to the separating element T for example by a means known to the person skilled in the art, for example by means of an adhesive, it being preferable to use epoxy resins, phenolic resins. Alternatively or additionally, the separating element T may also be shaped such that the respective ASC can be fitted or clamped into the separating element. This can already be implemented in a corresponding manner in the production of the dividing wall W.

In a preferred embodiment in which the dividing wall W comprises a separating element, this especially comprises a seal Di between separating element T and the ASCs (FIGS. 3 B, 3 C). This ensures in a particularly efficient manner that the dividing wall W is liquid-tight. The seal Di may be selected by the person skilled in the art for the respective ASC or the respective separating element T.

The seal Di especially comprises a material selected from the group consisting of elastomers, adhesives, preferably elastomers.

A useful elastomer is especially rubber, preferably ethylene-propylene-diene rubber (“EPDM”), fluoropolymer rubber (“FPM”), perfluoropolymer rubber (“FFPM”), acrylonitrile-butadiene rubber (“NBR”).

In a further preferred embodiment, the separating element T comprises at least two parts T₁ and T₂ that can be secured to one another and hence clamp the ASCs between them.

In this embodiment, it is then particularly preferable to mount a seal Di between separating element T and ASC in order to assure liquid-tightness.

The separating element T preferably comprises a material selected from the group consisting of plastic, glass, wood. More preferably, the separating element T consists of plastic. Even more preferably, the plastic is one selected from the group consisting of polypropylene, polystyrene, polyvinylchloride, post-chlorinated polyvinylchloride (“PVC-C”).

### 1.1.4.3 Frame Element R

In a further preferred embodiment, the dividing wall W also comprises a frame element R. The frame element R differs from the separating element T in that it is not disposed between the alkali metal cation-conducting solid-state electrolyte ceramics encompassed by the dividing wall W, i.e. does not separate these from one another. The frame element R especially bounds the surfaces O_{KK} and O_{A/MK} at least partly, preferably completely. What this means is more particularly: The frame element R surrounds the surfaces O_{KK} and O_{A/MK} at least partly, preferably completely.

The frame element R here may or may not be part of the surfaces O_{KK} and O_{A/MK}. The frame element R is preferably part of the surfaces O_{KK} and O_{A/MK}.

The frame element R is especially directly contactable or not directly contactable via the surfaces O_{KK} and O_{A/MK}, preferably directly contactable.

What is meant by “not directly contactable” with regard to the frame element R optionally encompassed by the dividing wall W is that the frame element R is formed exclusively as at least part of the surfaces of those sides of the dividing wall W which are not the sides S_{KK} and S_{A/MK}. More particularly, the frame element R in that case forms at least 1%, more preferably at least 25%, more preferably at least 50%, even more preferably 100%, of the surface areas of the sides of the dividing wall W that are not the sides S_{KK} and S_{A/MK}.

What is meant by “directly contactable” with regard to the frame element R optionally encompassed by the dividing wall W is that part of the surfaces O_{KK} and O_{A/MK} is formed by the surface of the frame element R, meaning that the frame element R encompassed by the dividing wall W is directly accessible at the two surfaces O_{KK} and O_{A/MK}, such that it can be wetted at the two surfaces O_{KK} and O_{A/MK}, for example, with aqueous solution, alcoholic solution, alcohol or water.

What this means in respect of the arrangement of the frame element R in the dividing wall W is that there is then a route from the surface O_{KK} on the S_{KK} side to the surface O_{A/MK} on the S_{A/MK} Side that leads completely through the frame element R.

This includes the following embodiments:
a portion of the edge of the surfaces O_{KK} and O_{A/MK} is formed by the frame element R (as shown in FIGS. 4 B, 4 D);
the edge of the surfaces O_{KK} and O_{A/MK} is formed completely by the frame element R (as shown in FIGS. 4 A, 4 C, 7 A, 7 B).

It is possible here for the frame element R additionally also to be formed as at least part of the surfaces of those sides of the dividing wall W that are not the sides S_{KK} and S_{A/MK}. More particularly, the frame element R forms at least 1%, more preferably at least 25%, more preferably at least 50%, even more preferably 100%, of the surface areas of the sides of the dividing wall W that are not the sides S_{KK} and S_{A/MK}.

FIG. 4 B and FIG. 4 D show, for example, embodiments in which the frame element R forms part of the surfaces of those sides of the dividing wall W that are not the sides S_{KK} and S_{A/MK}.

FIG. 4 A and FIG. 4 C show, for example, embodiments in which the frame element R completely forms the surfaces of those sides of the dividing wall W that are not the sides S_{KK} and S_{A/MK}.

The frame element R is especially made of a material selected from the group consisting of plastic, glass, wood. More preferably, the frame element R is made of plastic.

Even more preferably, the plastic is one selected from the group consisting of polypropylene, polystyrene, polyvinylchloride, PVC-C.

In a further preferred embodiment, when the dividing wall W comprises a separating element T and a frame element R, the frame element R and the separating element T are made of the same material, even more preferably both are made of plastic, which is even more preferably selected from polypropylene, polystyrene, polyvinylchloride, PVC-C.

The frame element R here may consist of one piece. In that case, the ASC is secured gaplessly to the frame element R for example by a means known to the person skilled in the art, for example by means of an adhesive, for which epoxy resins and phenolic resins are particularly suitable. Alternatively or additionally, the frame element R may also be shaped such that the respective ASC can be fitted or clamped into the frame element R.

This also means that, in the preferred embodiment in which the dividing wall W comprises at least two ASCs F_{A}, F_{B}, at least one separating element T and a frame element R, the ASCs, the at least one separating element T and the frame element R adjoin one another in a gapless manner. Thus, there then exist no gaps between separating element T, frame element R and the ASCs encompassed by the dividing wall W, through which glycol, glycolic solution, aqueous solution or water could flow from the S_{KK} side to the S_{A/MK} side or vice versa.

In addition, especially when the dividing wall W comprises at least two ASCs F_{A}, F_{B}, a frame element R and at least one separating element T, and the frame element R and the at least one separating element T are at least partly in one-piece form together with one another, the frame element R may consist of at least two parts that are secured to one another and clamp the ASCs between them. For example, the dividing wall W may then have a hinge by which the two parts of the frame element R can be opened and closed. In addition, the dividing wall W may then have a lock by which the two parts of the frame element R can be locked in place in the closed state (FIG. 7 A).

In the closed state, it is then possible for the ASCs and, if it is not already in one-piece form together with the frame element R, the separating element T to be clamped between the two parts of the frame element R. In this embodiment, it is then possible for a seal to be mounted between separating element T and ASC or frame element R and ASC, in order to assure liquid-tightness.

In a preferred embodiment, when the dividing wall W comprises at least two ASCs F_{A}, F_{B}, a frame element R and at least one separating element T, at least part of the separating element T is in one-piece form together with at least part of the frame element R. What this means more particularly is that, in that case, at least part of the separating element T merges into the frame element R.

Preferably, the at least one separating element T and the frame element R are then in one-piece form.

The embodiment of a frame element R has the advantage that it can function as part of the outer wall in the assembly of the electrolysis cell E. This part of the dividing wall W does not make contact with the solutions in the respective interior I_{KK}, I_{KA} Or I_{KM}, and it would therefore be a waste to take the at least one solid-state electrolyte ceramic F_{A} for this part. In addition, the part of the dividing wall W which is clamped between the outer wall or forms part thereof is subjected to pressures, which makes the brittle solid-state electrolyte ceramic F_{A} unsuitable. Instead, a fracture-resistant and cheaper material is thus selected for the frame R.

### 1.1.4.4 Production of the Dividing Wall W

The dividing wall W can be produced by methods known to the person skilled in the art.

The dividing wall W utilized in one embodiment of the method according to the invention may be an ASC F_{A} which is cut or formed by methods known to the person skilled in the art.

If the dividing wall W comprises a frame element R or at least one separating element T, the ASCs encompassed by the dividing wall may be placed into a casting mould, optionally together with seals, and the separating element may be cast using liquid plastic and then left to solidify (injection moulding method). In the course of solidification, this then surrounds the ASCs.

Alternatively, the separating element T is cast separately (or in parts) and then secured in a gapless manner (for example bonded) to the at least two ASCs.

### 1.1.4.5 Arrangement of the Dividing Wall W in the Electrolysis Cell E

1) The dividing wall W is arranged in the electrolysis cell E in such a way that the alkali metal cation-conducting solid-state electrolyte ceramic F_{A} encompassed by the dividing wall W makes direct contact with the interior I_{KK} on the S_{KK} side via the surface O_{KK}.

When the dividing wall W comprises at least two ASCs F_{A}, F_{B}, at least one separating element T and optionally a frame element R, the dividing wall W is arranged in the electrolysis cell E such that the alkali metal cation-conducting solid-state electrolyte ceramics F_{A} and F_{B} that are encompassed by the dividing wall W, and preferably also the separating element T, make direct contact with the interior I_{KK} on the S_{KK} side via the surface O_{KK}.

This means that the dividing wall W is arranged within the electrolysis cell E such that, when the interior I_{KK} on the S_{KK} side is completely filled with solution L₂, the solution L₂ then makes contact via the surface O_{KK} at least with the alkali metal cation-conducting solid-state electrolyte ceramic F_{A} encompassed by the dividing wall W, such that ions (e.g. alkali metal ions such as sodium, lithium) from F_{A} can enter the solution L₂.

When the dividing wall W comprises at least two ASCs F_{A}, F_{B}, at least one separating element T and optionally a frame element R, this means that the dividing wall W is arranged in the electrolysis cell E such that, when the interior I_{KK} on the S_{KK} side is completely filled with solution L₂, the solution L₂ then makes contact via the surface O_{KK} at least with the two alkali metal cation-conducting solid-state electrolyte ceramics F_{A} and F_{B} encompassed by the dividing wall W, and preferably also with the separating element T, such that ions (e.g. alkali metal ions such as sodium, lithium) from F_{A} and F_{B} can enter the solution L₂.

2) In addition, the dividing wall W, in the embodiments in which the electrolysis cell E does not comprise a middle chamber K_{M}, is arranged in the electrolysis cell E such that the alkali metal cation-conducting solid-state electrolyte ceramic F_{A} encompassed by the dividing wall W makes direct contact with the interior I_{KA} on the S_{A/MK} side via the surface O_{A/MK}.

When the dividing wall W comprises at least two ASCs F_{A}, F_{B}, at least one separating element T and optionally a frame element R, and when the electrolysis cell E does not comprise a middle chamber K_{M}, this means that the dividing wall W is arranged in the electrolysis cell E such that the alkali metal cation-conducting solid-state electrolyte ceramics encompassed by the dividing wall W, and preferably also the separating element T, make direct contact with the interior I_{KA} on the S_{A/MK} side via the surface O_{A/MK}.

What this means is as follows: in the embodiments in which the electrolysis cell E does not comprise a middle chamber K_{M}, the dividing wall W adjoins the interior I_{KA} of the anode chamber K_{A}.

In these embodiments, the dividing wall W is then arranged within the electrolysis cell E such that, when the interior I_{KA} on the S_{A/MK} side is completely filled with solution L₃, the solution L₃ then makes contact via the surface O_{A/MK} at least with the alkali metal cation-conducting solid-state electrolyte ceramic F_{A} encompassed by the dividing wall W, such that ions (e.g. alkali metal ions such as sodium, lithium) from the solution La can enter the ASC F_{A}.

When the dividing wall W comprises at least two ASCs F_{A}, F_{B}, at least one separating element T and optionally a frame element R, this then means that the dividing wall W is arranged in the electrolysis cell E such that, when the interior I_{KA} on the S_{A/MK} side is completely filled with solution L₃, the solution L₃ then makes contact via the surface O_{A/MK} at least with the two alkali metal cation-conducting solid-state electrolyte ceramics F_{A} and F_{B} encompassed by the dividing wall W, and preferably also with the separating element T, such that ions (e.g. alkali metal ions such as sodium, lithium) from the solution L₃ can enter the ASCs F_{A} and F_{B}.

3) In addition, the dividing wall W, in the cases in which the electrolysis cell E comprises at least one middle chamber K_{M}, is arranged in the electrolysis cell E such that the alkali metal cation-conducting solid-state electrolyte ceramic F_{A} encompassed by the dividing wall W makes direct contact with the interior I_{KM} on the S_{A/MK} side via the surface O_{A/MK}.

When the dividing wall W comprises at least two ASCs F_{A}, F_{B}, at least one separating element T and optionally a frame element R, and when the electrolysis cell E comprises at least one middle chamber K_{M}, this means that the dividing wall W is arranged in the electrolysis cell E such that the alkali metal cation-conducting solid-state electrolyte ceramics encompassed by the dividing wall W, and preferably also the separating element T, make direct contact with the interior I_{KM} on the S_{A/MK} side via the surface O_{A/MK}.

What this means is as follows: in the embodiments in which the electrolysis cell E comprises at least one middle chamber K_{M}, the dividing wall W adjoins the interior I_{KM} of the middle chamber K_{M}.

In these embodiments, the dividing wall W is then arranged within the electrolysis cell E such that, when the interior I_{KM} on the S_{A/MK} side is completely filled with solution L₃, the solution L₃ then makes contact via the surface O_{A/MK} at least with the alkali metal cation-conducting solid-state electrolyte ceramic F_{A} encompassed by the dividing wall W, such that ions (e.g. alkali metal ions such as sodium, lithium) from the solution L₃ can enter the ASC F_{A}.

When the dividing wall W comprises at least two ASCs F_{A}, F_{B}, at least one separating element T and optionally a frame element R, this then means that the dividing wall W is arranged in the electrolysis cell E such that, when the interior I_{KM} on the S_{A/MK} side is completely filled with solution L₃, the solution L₃ then makes contact via the surface O_{A/MK} at least with the two alkali metal cation-conducting solid-state electrolyte ceramics F_{A} and F_{B} encompassed by the dividing wall W, and preferably also with the separating element T, such that ions (e.g. alkali metal ions such as sodium, lithium) from the solution L₃ can enter the ASCs F_{A} and F_{B}.

In a preferred embodiment of the electrolysis cell E, at least 50%, especially at least 70%, preferably at least 90%, most preferably 100%, of the portion of the surface O_{KK} which is formed by ASCs makes contact with the interior I_{KK}.

In a preferred embodiment of the electrolysis cell E without a middle chamber, at least 50%, especially at least 70%, preferably at least 90%, most preferably 100%, of the portion of the surface O_{A/MK} which is formed by ASCs makes contact with the interior I_{KA}.

In a preferred embodiment of the electrolysis cell E with at least one middle chamber, at least 50%, especially at least 70%, preferably at least 90%, most preferably 100%, of the portion of the surface O_{A/MK} which is formed by ASCs makes contact with the interior I_{KM}.

### 1.2 Step (a) of the Method According to the Invention

Step (a) of the method according to the invention relates to the preparation of a solution L₁ of M_{A} glycolate in glycol, where M_{A} is an alkali metal cation. The method is conducted in an electrolysis cell E.

M_{A} is preferably selected from the group consisting of Li⁺, K⁺, Na⁺, more preferably from the group consisting of K⁺, Na⁺. Most preferably, M_{A}=Na⁺.

1.2.1 Process according to the invention in an electrolysis cell E without a middle chamber K_{M} In the cases in which the electrolysis cell E does not comprise a middle chamber K_{M}, the steps (α1), (α2), (α3) that proceed simultaneously are performed.

### 1.2.1.1 Step (α1)

In step (α1), a solution L₂ comprising glycol, preferably comprising an alkali metal glycolate M_{A} glycolate and glycol, is directed through I_{KK}.

Solution L₂ is preferably free of water. What is meant in accordance with the invention by “free of water” is that the weight of water in solution L₂ based on the weight of the glycol in solution L₂ (mass ratio) is ≤1:10, more preferably ≤1:20, even more preferably ≤1:100, even more preferably ≤0.5:100, even more preferably ≤1:1000, even more preferably ≤1:10 000.

If solution L₂ comprises M_{A} glycolate, the proportion by mass of M_{A} glycolate in solution L₂, based on the overall solution L₂, is especially >0% to 30% by weight, preferably 0.1% to 20% by weight, more preferably 0.2% to 10% by weight, more preferably 0.5% to 5% by weight, most preferably 0.7% to 2% by weight, at the very most preferably 1% by weight.

If solution L₂ comprises M_{A} glycolate, the mass ratio of M_{A} glycolate to glycol in solution L₂ is especially in the range of 1:1000 to 1:5, more preferably in the range of 1:250 to 3:20, even more preferably in the range of 1:120 to 1:8, even more preferably 1:100.

### 1.2.1.2 Step (α2)

In step (α2), a neutral or alkaline, aqueous solution L₃ of a salt S comprising M_{A} as cation is directed through I_{KA}.

The salt S is preferably a halide, sulfate, sulfite, nitrate, hydrogen carbonate or carbonate of M_{A}, even more preferably a halide.

Halides are fluorides, chlorides, bromides, iodides. The most preferred halide is chloride.

The pH of the aqueous solution L₃ is >7.0, preferably in the range of 7 to 12, more preferably in the range of 8 to 11, even more preferably 10 to 11, most preferably 10.5.

The proportion by mass of salt S in solution L₃ is preferably in the range of >0% to 20% by weight, preferably 1% to 20% by weight, more preferably 5% to 20% by weight, even more preferably 10% to 20% by weight, most preferably 20% by weight, based on the overall solution L₃.

### 1.2.1.3 Step (α3)

In step (α3), a voltage is then applied between E_{A} and E_{K}.

This results in a transfer of current from the charge source to the anode, a transfer of charge via ions to the cathode and finally a transfer of current back to the charge source. The charge source is known to the person skilled in the art and is typically a rectifier that converts alternating current into direct current and can generate particular voltages via voltage transformers.

This in turn has the following consequences:
the solution Li is obtained at the outlet A_{KK}, the concentration of M_{A} glycolate being higher in L₁ than in L₂,
an aqueous solution L₄ of S is obtained at the outlet A_{KA}, the concentration of S being lower in L₄ than in L₃.

In step (α3) of the method according to the invention, in particular, such a voltage is applied that such a current flows that the current density (=ratio of the current supplied to the electrolysis cell to the area of the solid-state electrolyte in contact with the anolyte present in I_{KA}) is in the range from 10 to 8000 A/m², more preferably in the range from 100 to 2000 A/m², even more preferably in the range from 300 to 800 A/m², and even more preferably is 494 A/m². This can be determined in a standard manner by the person skilled in the art. The area of the solid-state electrolyte in contact with the anolyte present in the interior I_{KA} of the anode chamber K_{A} is especially 0.00001 to 10 m², preferably 0.0001 to 2.5 m², more preferably 0.0002 to 0.15 m², even more preferably 2.83 cm².

It will be apparent that step (α3) of the method according to the invention is conducted when the interior I_{KA} of the anode chamber K_{A} is at least partly laden with L₃ and the interior I_{KK} of the cathode chamber K_{K} is at least partly laden with L₂, such that both L₃ and L₂ make contact with the ASCs encompassed by the dividing wall W and especially also with the separating element T when the dividing wall W comprises one.

The fact that a transfer of charge between E_{A} and Ex takes place in step (α3) implies that I_{KK} and I_{KA} are at the same time laden with L₂ and L₃ respectively such that they cover the electrodes E_{K}/E_{A} to such an extent that the circuit is complete.

This is the case especially when a liquid stream of L₃ is directed continuously through I_{KA} and a liquid stream of L₂ through I_{KK}, and the liquid stream of L₃ covers electrode E_{A} and the liquid stream of L₂ covers electrode E_{K} at least partly, preferably completely.

In a further preferred embodiment, the method according to the invention is performed continuously, i.e. step (α1) and step (α2) are performed continuously, while applying voltage as per step (α3).

After performance of step (α3), solution L₁ is obtained at the outlet A_{KK}, the concentration of M_{A} glycolate being higher in L₁ than in L₂. If L₂ already comprised M_{A} glycolate, the concentration of M_{A} glycolate in L₁ is preferably 1.01 to 200.2 times, more preferably 5.04 to 100.8 times, even more preferably 10.077 to 50.4 times, even more preferably 18.077 to 20.08 times, higher than in L₂, most preferably 20.00 times higher than in L₂, where the proportion by mass of M_{A} glycolate in L₁ and in L₂ is more preferably in the range from 0.1% to 50% by weight, even more preferably 1% to 20% by weight.

An aqueous solution L₄ of S is obtained at the outlet A_{KA}, the concentration of S being lower in L₄ than in L₃.

The concentration of the cation M_{A} in the aqueous solution L₃ is preferably in the range of 0.5 to 5 mol/l, more preferably 1 mol/l. The concentration of the cation M_{A} in the aqueous solution La is more preferably 0.5 mol/l lower than that of the aqueous solution L₃ used in each case.

More particularly, steps (α1) to (α3) of the method according to the invention are conducted at a temperature of 20° C. to 110° C., preferably 50° C. to 105° C., more preferably 80° C. to 99° C., even more preferably 90° C. to 95° C., and at a pressure of 0.5 bar to 1.5 bar, preferably 0.9 bar to 1.1 bar, more preferably 1.0 bar.

In the course of performance of steps (α1) to (α3) of the method according to the invention, hydrogen is typically formed in I_{KK}, which can be removed from the cell together with solution Li via the outlet A_{KK}. The mixture of hydrogen and solution Li can then, in a particular embodiment of the present invention, be separated by methods known to the person skilled in the art. When the alkali metal compound used is a halide, especially chloride, it is possible for chlorine or another halogen gas to form in I_{KA}, and this can be removed from the cell together with solution La via the outlet A_{KK}.

In addition, it is also possible for oxygen or/and carbon dioxide to form, which can likewise be removed. The mixture of chlorine, oxygen and/or CO₂ and solution La can then, in a particular embodiment of the present invention, be separated by methods known to the person skilled in the art. It is then likewise possible, after the chlorine, oxygen and/or CO₂ gases have been separated from solution L₄, to separate these by methods known to the person skilled in the art.

1.2.2 Process According to the Invention in an Electrolysis Cell E with a Middle Chamber K_{M}

In the cases in which the electrolysis cell E comprises at least one middle chamber K_{M}, the steps (31), (2), (β3) that proceed simultaneously are performed.

It is preferable that the electrolysis cell E comprises at least one middle chamber K_{M}, and then the steps (β1), (β2), (β3) that proceed simultaneously are performed.

### 1.2.2.1 Step (β1)

In step (31), a solution L₂ comprising glycol, preferably comprising an alkali metal glycolate M_{A} and glycol, is directed through I_{KK}.

Solution L₂ is preferably free of water. What is meant in accordance with the invention by “free of water” is that the weight of water in solution L₂ based on the weight of the glycol in solution L₂ (mass ratio) is ≤1:10, more preferably ≤1:20, even more preferably ≤1:100, even more preferably ≤0.5:100.

If solution L₂ comprises M_{A} glycolate, the proportion by mass of M_{A} glycolate in solution L₂, based on the overall solution L₂, is especially >0% to 30% by weight, preferably 0.1% to 20% by weight, more preferably 0.2% to 20% by weight, more preferably 0.5% to 5% by weight, most preferably 0.7% to 2% by weight, at the very most preferably 1% by weight.

If solution L₂ comprises M_{A} glycolate, the mass ratio of M_{A} glycolate to glycol in solution L₂ is especially in the range of 1:1000 to 1:5, more preferably in the range of 1:250 to 3:20, even more preferably in the range of 1:120 to 1:8, even more preferably 1:100.

### 1.2.2.2 Step (32)

In step (β2), a neutral or alkaline, aqueous solution L₃ of a salt S comprising M_{A} as cation is directed through I_{KM}, then via V_{AM}, then through I_{KA}.

The salt S is preferably a halide, sulfate, sulfite, nitrate, hydrogen carbonate or carbonate of M_{A}, even more preferably a halide.

Halides are fluorides, chlorides, bromides, iodides. The most preferred halide is chloride.

The pH of the aqueous solution L₃ is >7.0, preferably in the range of 7 to 12, more preferably in the range of 8 to 11, even more preferably 10 to 11, most preferably 10.5.

The proportion by mass of salt S in solution L₃ is preferably in the range of >0% to 20% by weight, preferably 1% to 20% by weight, more preferably 5% to 20% by weight, even more preferably 10% to 20% by weight, most preferably 20% by weight, based on the overall solution L₃.

### 1.2.2.3 Step (β3)

In step (β3), a voltage is then applied between E_{A} and E_{K}.

This results in a transfer of current from the charge source to the anode, a transfer of charge via ions to the cathode and finally a transfer of current back to the charge source. The charge source is known to the person skilled in the art and is typically a rectifier that converts alternating current into direct current and can generate particular voltages via voltage transformers.

This in turn has the following consequences:
the solution L₁ is obtained at the outlet A_{KK}, the concentration of M_{A} glycolate being higher in L₁ than in L₂,
an aqueous solution L₄ of S is obtained at the outlet A_{KA}, the concentration of S being lower in L₄ than in L₃.

In step (β3) of the method according to the invention, in particular, such a voltage is applied that such a current flows that the current density (=ratio of the current supplied to the electrolysis cell to the area of the solid-state electrolyte in contact with the anolyte present in I_{KM}) is in the range from 10 to 8000 A/m², more preferably in the range from 100 to 2000 A/m², even more preferably in the range from 300 to 800 A/m², and even more preferably is 494 A/m². This can be determined in a standard manner by the person skilled in the art. The area of the solid-state electrolyte in contact with the anolyte present in the middle chamber K_{M} is especially 0.00001 to 10 m², preferably 0.0001 to 2.5 m², more preferably 0.0002 to 0.15 m², even more preferably 2.83 cm².

It will be apparent that step (β3) of the method according to the invention is conducted when the interiors I_{KA} and I_{KM} of the two chambers K_{M} and K_{A} are at least partly laden with L₃ and the interior I_{KK} is at least partly laden with L₂, such that both L₃ and L₂ make contact with the solid-state electrolytes encompassed by the dividing wall W and especially also with the separating element T when the dividing wall W comprises one.

The fact that a transfer of charge takes place between E_{A} and E_{K} in step (β3) implies that I_{KK}, I_{KM} and I_{KA} are simultaneously laden with L₂ and L₃ respectively such that they cover the electrodes E_{K}/E_{A} to such an extent that the circuit is complete.

This is the case especially when a liquid stream of L₃ is directed continuously through I_{KM}, V_{AM} and I_{KA} and a liquid stream of L₂ through I_{KK}, and the liquid stream of L₃ covers electrode E_{A} and the liquid stream of L₂ covers electrode E_{K} at least partly, preferably completely.

In a further preferred embodiment, the method according to the invention is performed continuously, i.e. step (31) and step (β2) are performed continuously, while applying voltage as per step (β3).

After performance of step (β3), solution L₁ is obtained at the outlet A_{KK}, the concentration of M_{A} glycolate being higher in L₁ than in L₂. If L₂ already comprised M_{A} glycolate, the concentration of M_{A} glycolate in Li is preferably 1.01 to 200.2 times, more preferably 5.04 to 100.80 times, even more preferably 10.077 to 50.40 times, even more preferably 18.077 to 20.08 times, higher than in L₂, most preferably 20.00 times higher than in L₂, where the proportion by mass of M_{A} glycolate in L₁ and in L₂ is more preferably in the range from 0.1% to 50% by weight, even more preferably 1% to 20% by weight.

An aqueous solution L₄ of S is obtained at the outlet A_{KA}, the concentration of S being lower in L₄ than in L₃.

The concentration of the cation M_{A} in the aqueous solution L₃ is preferably in the range of 0.5 to 5 mol/l, more preferably 1 mol/l. The concentration of the cation M_{A} in the aqueous solution L₄ is more preferably 0.5 mol/l lower than that of the aqueous solution L₃ used in each case.

More particularly, steps (β1) to (β3) of the method according to the invention are conducted at a temperature of 20° C. to 110° C., preferably 50° C. to 105° C., more preferably 80° C. to 99° C., even more preferably 90° C. to 95° C., and at a pressure of 0.5 bar to 1.5 bar, preferably 0.9 bar to 1.1 bar, more preferably 1.0 bar.

In the course of performance of steps (31) to (3) of the method according to the invention, hydrogen is typically formed in the cathode chamber I_{KK}, which can be removed from the cell together with solution L₁ via the outlet A_{KK}. The mixture of hydrogen and solution L₁ can then, in a particular embodiment of the present invention, be separated by methods known to the person skilled in the art. When the alkali metal compound used is a halide, especially chloride, it is possible for chlorine or another halogen gas to form in I_{KA}, and this can be removed from the cell together with solution La via outlet A_{KK}. In addition, it is also possible for oxygen or/and carbon dioxide to form, which can likewise be removed. The mixture of chlorine, oxygen and/or CO₂ and solution La can then, in a particular embodiment of the present invention, be separated by methods known to the person skilled in the art. It is then likewise possible, after the chlorine, oxygen and/or CO₂ gases have been separated from solution L₄, to separate these by methods known to the person skilled in the art.

### 1.2.2.4 Additional Advantages of Steps (31) to (β3)

This performance of steps (1) to (β3) brings yet further surprising advantages that were not to be expected in the light of the prior art. Steps (31) to (β3) of the method according to the invention protect the acid-labile solid-state electrolyte from corrosion without, as in the prior art, having to sacrifice alkoxide solution from the cathode space as buffer solution. Thus, the method according to the invention is more efficient than the procedure described in WO 2008/076327 A1, in which the product solution is used for the middle chamber, which reduces the overall conversion.

2. Step b): Reaction of PET with the Solution L₁<21>

In step (b) of the method according to the invention, the solution L₁<21> obtained in step (a), comprising glycol and M_{A} glycolate, is reacted with PET to give a mixture M₁ comprising BHET.

### 2.1 PET Starting Material

The PET which is used in step (b) of the method according to the invention may be any PET which has to be depolymerized. Typically, such PET occurs as waste, especially in the home, in industry or agriculture.

In one embodiment of the method according to the invention, the PET to be depolymerized is thus in a mixture with other plastics, especially at least one plastic selected from polyethylene (“PE”), polyvinylchloride (“PVC”). This is typically the case when PET from plastic wastes is to be depolymerized in the method according to the invention. In this embodiment, the PET is at least partly separated from the other plastics, preferably by sorting, before being subjected to step (b) of the method according to the invention.

In one embodiment of the method according to the invention, the PET is subjected to at least one pretreatment step.

Such pretreatment steps are described, for example, in DE 10032899 C2.

According to the invention, the PET is subjected to at least one pretreatment step selected from chemical pretreatment step, comminution step, before being used in step (b).

In the cases in which the PET is in a mixture with other plastics, the PET is preferably subjected to at least one pretreatment step selected from at least partial separation from other plastics, preferably by sorting, chemical pretreatment step, comminution step, before being used in step (b).

In the cases in which the PET is in a mixture with other plastics, the PET is more preferably first separated at least partly from other plastics, then subjected to at least one chemical pretreatment and finally comminuted.

The chemical pretreatment step is especially a washing step. Such a washing step has the advantage that any impurities, especially food residues, residues of cosmetics and/or bodily secretions (e.g. blood, sperm, faeces), are removed prior to the performance of step (b). Such impurities can lower the efficiency of the reaction in step (b) and/or worsen the purity of the BHET thus obtained.

In the chemical pretreatment step, especially the wash step, the waste is especially heated in a wash solution at a temperature of 30° C. to 99° C., preferably 50° C. to 90° C., more preferably 70° C. to 85° C.

Typical wash solutions are familiar to the person skilled in the art and are preferably selected from:
aqueous solution of a surfactant, preferably a nonionic surfactant;
aqueous solution of an alkali metal hydroxide or alkaline earth metal hydroxide, preferably aqueous NaOH.

The treatment time in the chemical pretreatment step, especially the wash step, is especially 1 min to 12 h, preferably 10 min to 6 h, more preferably 30 min to 2 h, even more preferably 45 to 90 min, most preferably 60 min.

After the treatment of the PET by the chemical pretreatment step, especially the wash step, the aqueous solution is separated off, for example by filtration, and the cleaned PET is preferably washed once with water in order to remove residues of the wash solution.

The PET waste thus obtained is then dried, especially in a drying cabinet.

The temperature used for drying here is especially in the range of 30 to 120° C., preferably 50° C. to 100° C., more preferably 60° C. to 90° C., most preferably 80° C.

The comminution step has the advantage that the surface area of the PET available for the reaction in step (b) is increased. This increases the reaction rate of the reaction in step (b). The comminution can be effected in apparatuses known to the person skilled in the art, for example a shredder or a cutting mill.

In a further embodiment of the method according to the invention, the PET is decolorized or coloured in a controlled manner before being subjected to step (b). This can be conducted by methods known to the person skilled in the art, for example decolorization with hydrogen peroxide or dyeing with a dye.

### 2.2 Reaction Conditions

The reaction of the PET with a solution L₁<21> comprising glycol and M_{A} glycolate to give a mixture M₁ can then be effected under the conditions that are familiar to the person skilled in the art.

Preferably, the reaction in step (b) is conducted until, i.e. up to a juncture t_{b} at which, at least P=10%, preferably at least P=20%, more preferably at least P=25%, more preferably at least P=30%, more preferably at least P=40%, more preferably at least P=50%, more preferably at least P=60%, more preferably at least P=70%, more preferably at least P=80%, more preferably at least P=90%, more preferably at least P=95%, even more preferably at least P=99%, of the PET used in step (b) has been converted.

This percentage P is calculated by the following formula:

$$P = ( {n}_{TS} + {n}_{MHET} + {n}_{BHET} ) / {n}_{PET} .$$

n_{PET} here is the molar amount of repeat units of the following structure ( [symbol] ) in the PET used in step (b):

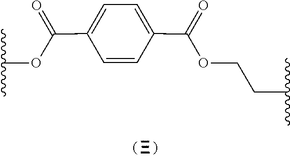

n_{TS} is the molar amount of TS formed in step (b) from commencement of step (b) up to the juncture t_{b}.

n_{MHET} is the molar amount of MHET formed in step (b) from commencement of step (b) up to the juncture t_{b}.

n_{BHET} is the molar amount of BHET formed in step (b) from commencement of step (b) up to the juncture t_{b}.

The structures of compounds BHET, MHET, TS are as follows:

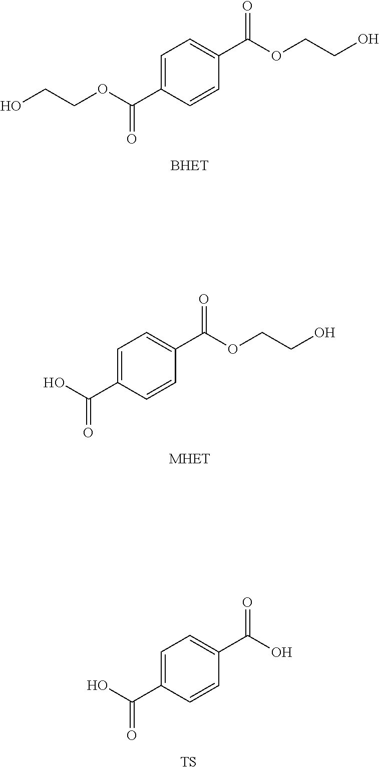

“MHET” also encompasses the corresponding carboxylate of the structure shown.

“TS” also encompasses the corresponding mono- and dicarboxylate of the structure shown.

The reaction in step (b) is especially conducted at a temperature of at least 100° C., preferably at a temperature in the range from ≥100° C. to ≤197° C., more preferably at a temperature in the range from ≥130° C. to ≤197° C., more preferably at a temperature in the range from ≥150° C. to ≤197° C., more preferably at a temperature in the range from ≥175° C. to ≤197° C.

The reaction in step (b) is preferably conducted at the boiling temperature of the glycol. Even more preferably, glycol is refluxed, meaning that glycol is evaporated out of the reaction, condenses and is then returned to the reaction. This refluxing can be established by means familiar to the person skilled in the art, for example in a distillation apparatus.

The total weight of the M_{A} glycolate used in the method based on the total weight of the PET used in the method is especially in the range from 0.1% to 100% by weight, preferably in the range from 0.5% to 80% by weight, more preferably in the range from 1.0% to 50% by weight, more preferably in the range from 1.5% to 25% by weight, more preferably in the range from 2.0% to 10% by weight, more preferably in the range from 2.5% to 6.0% by weight, more preferably 3.5% to 5.0% by weight, most preferably 3.9% by weight.

The reaction can be effected with devices familiar to the person skilled in the art.

After step (b) of the method according to the invention has ended, a mixture M₁ is obtained, in which the molar ratio n of the molar amount of BHET (n_{BHET}) to the sum total of the molar amounts of MHET and TS (n_{MHET}+n_{TS}) is in the range of 1:1 to 1000:1, preferably 2:1 to 500:100, more preferably 4:1 to 300:1, even more preferably 10:1 to 100:1, yet more preferably 13:1 to 60:1, yet more preferably 13:1 to 24:1.

$$η = {n}_{BHET} / ( {n}_{MHET} + {n}_{TS} )$$

### 2.3 Preferred Step (c)

In a preferred further step (c), BHET is at least partly separated from M₁. This is even more preferably effected by crystallization and/or distillation. Even more preferably, BHET in step (c) is filtered out of M₁ and then crystallized.

### 3. Process for Recycling of PET

The BHET obtained in the mixture M₁ in the method according to the invention is preferably polymerized to PET in a method of recycling of polyethylene in a step (2).

This polymerization is known to the person skilled in the art as “polycondensation” and is described, for example, in EP 0 723 951 A1 and by Th. Rieckmann and S. Völker in chapter 2 “Poly (Ethylene Terephthalate) Polymerization—Mechanism, Catalysis, Kinetics, Mass Transfer and Reactor Design” on page 2 of the book “Modern Polyesters: Chemistry and Technology of Polyesters and Copolyesters. Edited by J. Scheirs and T. E. Long, 2003, John Wiley & Sons, Ltd ISBN: 0-471-49856-4”.

In particular, for this purpose, BHET is polymerized back to PET in step (ζ) in the presence of catalysts, which are especially catalysts selected from the group consisting of antimony compounds, preferably Sb₂O₃.

Preferably, the polymerization of BHET to PET in step (ζ) is conducted at least at the boiling temperature of the glycol. In particular, during the polymerization in step (ζ), glycol is removed from the reaction mixture in order to shift the reaction equilibrium to the side of the polymer PET.

More preferably, the polymerization of BHET to PET in step (ζ) is conducted at the boiling temperature of the glycol. Even more preferably, in that case, during the polymerization in step (ζ), glycol is removed from the reaction mixture in order to shift the reaction equilibrium to the side of the polymer PET.

This is especially achieved by distillation at a pressure of <1 bar, preferably 0.1 mbar, at the respective boiling temperature of the glycol at the respective pressure.

### EXAMPLES

### 1. Inventive Example E1

### 1.1 Preparation of the Glycolic Sodium Glycolate Solution by Electrolysis

### 1.1.1 Experimental Setup

The electrolytic recovery of sodium glycolate was conducted in a three-chamber electrolysis cell.

The middle chamber was divided from the anode chamber by a filter cloth and from the cathode chamber by a 15×15 cm Nasicon ceramic.

The anode used was a DSA [“dimensionally stable anode”; ruthenium oxide/iridium oxide-coated titanium anode (RuO₂+IrO₂/Ti)]; the cathode consisted of VA steel (VA means “corrosion-resistant”; stainless steel).

A Gamry Reference 3000 (AE) potentiostat and Reference 30K Booster were used as power source.

The cathode chamber of the electrolysis cell was connected to a 250 ml heatable jacketed vessel with magnetic stirrer, from which it was possible to pump electrolyte by means of a peristaltic pump through a conductivity measurement point and a further 100 ml glass heat exchanger into the cathode chamber of the electrolysis cell. Thence it was possible to pump the catholyte back into the jacketed vessel. The catholyte was accordingly circulated.

A thermostat with PT 100 sensor (platinum sensor having a nominal resistance of 100Ω at a temperature of 0° C.) and a heat exchanger were used to measure and adjust the temperature of the electrolyte on the cathode side.

The middle chamber of the electrolysis cell was connected to a reservoir vessel from which a peristaltic pump was used to pump electrolyte into the middle chamber, then via the filter cloth into the anode chamber, and thence via a pH measurement point into a collecting vessel.

A thermostat with PT 100 sensor or heat exchanger was used to measure and adjust the temperature of the electrolyte on the anode side. The anolyte was not circulated.

### 1.1.2 Experimental Procedure:

The thermostat for the cathode side was adjusted to 90° C. and started. 650 g of 1% by weight sodium glycolate solution was introduced into the heatable jacketed vessel, and the peristaltic pump was started (delivery rate 1000 ml/h), thus pumping the sodium glycolate solution through a conductivity measurement point and a further heat exchanger into the cathode chamber of the electrolysis cell. The glycolate then flowed out of the cathode chamber back into the jacketed vessel. The glycolate and the chamber were thus kept at a controlled temperature of 90° C. The thermostat for the NaCl side was adjusted to 105° C. and the peristaltic pump for the NaCl brine was started (delivery rate 4000 ml/h). The 20% by weight NaCl brine at pH 11 was pumped from a reservoir vessel through a heat exchanger into the middle chamber of the electrolysis cell. Thence it flowed through the filter cloth into the anode chamber and then out of the cell into a pH measurement point and then into the collecting vessel. The NaCl brine was not circulated. Once the cathode chamber had been heated to 90° C. and the anode side at the pH measurement point was at a temperature of 80° C., the electrolysis cell current was switched on. For this purpose, the potentiostat was switched to galvanostatic operation. The current was fixed at 10 amperes and the voltage was controlled correspondingly. Then the conductivity measurement for the glycolate and the pH measurement of the NaCl brine were recorded.

Every 20 seconds, current, voltage, glycolate temperature, glycolate conductivity, brine pH and brine temperature were recorded. The hydrogen formed and the chlorine formed were removed by suction. The chlorine was neutralized with NaOH in gas scrubbing bottles.

The electrolysis was conducted for 4 h. Then the current was switched off and the cell was completely emptied.

The sodium glycolate solution had a concentration of ˜ 20% by weight.

1.2 PET Depolymerization with Glycolic Sodium Glycolate Solution from the Electrolysis

In the method according to the invention, an autoclave was initially charged with 100 g of PET together with 800 g of ethylene glycol. The solution was then heated to 150° C. while stirring. As soon as the temperature of 150° C. had been attained, 19.5 g of 20% sodium glycolate solution in ethylene glycol (corresponding to 0.046 mol) from the electrolysis was added. The reaction was conducted over the course of five hours, and the reactor output was analysed after cooling. The resultant conversion of BHET (1) and mono-2-hydroxyethylterephthalic acid (=“MHET”) (2) and terephthalic acid (=“TS”) (3) is shown in FIG. 8 (determined by gas chromatography; in % conversion based on the repeat unit of the structure (E) of the PET used; sparse hatching “////”).

### 2. Comparative Example V1

In a comparative experiment, an autoclave was initially charged with 100 g of PET together with 800 g of ethylene glycol. The solution was then heated to 150° C. while stirring. The reaction is conducted over the course of five hours, and the reactor output is analysed after cooling. The resultant conversion of BHET (1) and MHET (2) and of TS (3) is shown in FIG. 8 (black, “▪”).

### 3. Comparative Example V2

In a comparative experiment, an autoclave is initially charged with 100 g PET together with 800 g of ethylene glycol. The solution was then heated to 150° C. while stirring. As soon as the temperature of 150° C. had been attained, 3.7 g of 50% NaOH solution in water (corresponding to 0.046 mol) was added. The reaction was conducted over the course of five hours, and the reactor output was analysed after cooling. The resultant conversion of BHET (1) and MHET (2) and of TS (3) is shown in FIG. 8 (bold hatching: “////”).

### 4. Result

Comparison of the content of BHET, MHET and TS in the depolymerized product in Inventive Example E1 and Comparative Examples V1, V2 (see FIG. 8) shows that the depolymerization using the glycolic sodium glycolate solution obtained electrolytically affords a higher proportion of BHET. This is advantageous since more product is available as a result, which can be converted directly in a polycondensation to new PET product.

6. Reference symbols in the figures

| | | |
|---|---|---|
| Electrolysis cell | E | <1> |
| Anode chamber | K_{A} | <11> |
| Inlet | Z_{KA} | <110> |
| Outlet | A_{KA} | <111> |
| Interior | I_{KA} | <112> |
| Anodic electrode | E_{A} | <113> |
| Cathode chamber | K_{K} | <12> |
| Inlet | Z_{KK} | <120> |
| Outlet | A_{KK} | <121> |
| Interior | I_{KK} | <122> |
| Cathodic electrode | E_{K} | <123> |
| Middle chamber | K_{M} | <13> |
| Inlet | Z_{KM} | <130> |
| Outlet | A_{KM} | <131> |
| Interior | I_{KM} | <132> |
| Diffusion barrier | D | <14> |
| Connection | V_{AM} | <15> |
| Dividing wall | W | <16> |
| Side | S_{KK} | <161> |
| Side | S_{A/MK} | <162> |
| Surface | O_{KK} | <163> |
| Surface | O_{A/MK} | <164> |
| Separating element | T | <17> |
| Part of a separating element | | <171> |
| Part of a separating element | | <172> |
| Solid-state electrolyte ceramic | F_{A} | <18> |
| Solid-state electrolyte ceramic | F_{B} | <19> |
| Solid-state electrolyte ceramic | F_{C} | <28> |
| Solid-state electrolyte ceramic | F_{D} | <29> |
| Solid-state electrolyte ceramics | F_{E}, F_{F}, F_{G}, F_{H}, F_{I} | <30>, <31>, <32>, |
| | | <33>, <34> |
| Frame element | R | <20> |
| Frame part | | <201> |
| Frame part | | <202> |
| Seal | Di | <40> |
| Hinge | | <50> |
| Lock | | <60> |
| Outer wall | W_{A} | <80> |
| Solution comprising M_{A} glycolate in glycol | L₁ | <21> |
| Solution comprising glycol | L₂ | <22> |
| Neutral or alkaline, aqueous solution of a salt S | L₃ | <23> |
| comprising M_{A} as cation | | |
| Aqueous solution of S, where [S]_{L4} < [S]_{L3} | L₄ | <24> |

1-15. (canceled)

16. A method for depolymerizing polyethylene terephthalate (PET), comprising the following steps:
(a) producing a solution L₁<21> of M_{A} glycolate in glycol, wherein M_{A} is an alkali metal cation, in an electrolysis cell E<1> comprising:
at least one anode chamber K_{A}<11> having at least one inlet Z_{KA}<110>, at least one outlet A_{KA}<111>, and an interior I_{KA}<112> comprising an anodic electrode E_{A}<113>;
at least one cathode chamber K_{K}<12> having at least one inlet Z_{KK}<120>, at least one outlet A_{KK}<121>, and an interior I_{KK}<122> comprising a cathodic electrode E_{K}<123>;
optionally at least one interposed middle chamber K_{M}<13> having at least one inlet Z_{KM}<130>, at least one outlet A_{KM}<131> and an interior I_{KM}<132>; wherein I_{KA}<112> and I_{KM}<132> are divided from one another by a diffusion barrier D<14>, and A_{KM}<131> is connected by a connection V_{AM}<15> to the inlet Z_{KA}<110>, such that liquid can be passed from I_{KM}<132> into I_{KA}<112> via the connection V_{AM}<15>;
wherein:
in cases in which the electrolysis cell E<1> does not comprise a middle chamber K_{M}<13>, I_{KA}<112> and I_{KK}<122> are divided from one another by a dividing wall W<16>;
in the cases in which the electrolysis cell E<1> comprises at least one middle chamber K_{M}<13>, I_{KK}<122> and I_{KM}<132> are divided from one another by a dividing wall W<16>;
wherein the dividing wall W<16> has one side S_{KK}<161> having the surface O_{KK}<163> and a side S_{A/MK}<162> which is on the opposite side from the S_{KK}<161> side and has the surface O_{A/MK}<164>, wherein the dividing wall W<16> comprises at least one alkali metal cation-conducting solid-state electrolyte ceramic F_{A}<18> in such a way that the alkali metal cation-conducting solid-state electrolyte ceramic F_{A}<18> encompassed by the dividing wall W<16> makes direct contact with the interior I_{KK}<122> on the S_{KK}<161> side via the surface O_{KK}<163>;
and wherein
in cases in which the electrolysis cell E<1> does not comprise a middle chamber K_{M}<13>, the alkali metal cation-conducting solid-state electrolyte ceramic F_{A}<18> encompassed by the dividing wall W<16> makes direct contact with the interior I_{KA}<112> on the S_{A/MK}<162> side via the surface O_{A/MK}<164>;
in the cases in which the electrolysis cell E<1> comprises at least one middle chamber K_{M}<13>, the alkali metal cation-conducting solid-state electrolyte ceramic F_{A}<18> encompassed by the dividing wall W<16> makes direct contact with the interior I_{KM}<132> on the S_{A/MK}<162> side via the surface O_{A/MK}<164>;
(α) wherein, in the electrolysis cell E<1>, when it does not comprise a middle chamber K_{M}<13>, the following steps (α1), (α2), (α3) that proceed simultaneously are performed:
(α1) a solution L₂<22> comprising glycol is directed through I_{KK}<122>;
(α2) a neutral or alkaline, aqueous solution L₃<23> of a salt S comprising M_{A} as cation is directed through I_{KA}<112>;
(α3) voltage is applied between E_{A}<113> and E_{K}<123>;
or
(β) wherein, in the electrolysis cell E<1>, when it comprises at least one middle chamber K_{M}<13>, the following steps (β1), (β2), (3) that proceed simultaneously are performed:
(β1) a solution L₂<22> comprising glycol is directed through I_{KK}<122>;
(β2) a neutral or alkaline, aqueous solution L₃<23> of a salt S comprising M_{A} as cation is directed through I_{KM}<132>, then through V_{AM}<15>, then through I_{KA}<112>;
(β3) voltage is applied between E_{A}<113> and E_{K}<123>;
which affords the solution L₁<21> at the outlet A_{KK}<121>, the concentration of M_{A} glycolate being higher in L₁<21> than in L₂<22>;
and which affords an aqueous solution L₄<24> of S at the outlet A_{KA}<111>, the concentration of S being lower in L₄<24> than in L₃<23>;
(b) reacting the solution L₁<21> with PET to give a mixture M₁ comprising bis-2-hydroxyethyl terephthalate (BHET).

17. The method of claim 16, wherein the alkali metal cation-conducting solid-state electrolyte ceramic F_{A}<18> has a structure of the formula M^{I}_{1+2w+x-y+z}M^{II}_{w}M^{III}ₓZr^{IV}_{2-w-x-y}M^{V}_{y}(SiO₄)_{z}(PO₄)_{3-z};
wherein:
M^{I} is either Na⁺ or Li⁺;
M^{II} is a divalent metal cation;
M^{III} is a trivalent metal cation;
M^{V} is a pentavalent metal cation;
the Roman indices I, II, III, IV, V indicate the oxidation numbers in which the respective metal cations exist;
and w, x, y, z are real numbers, wherein 0≤x<2, 0≤y<2, 0≤w<2, 0≤z<3, and wherein w, x, y, z are chosen such that 1+2w+x−y+z≥0 and 2−w−x−y≥0.

18. The method of claim 16, wherein the electrolysis cell E<1> does not comprise a middle chamber K_{M}<13>.

19. The method of claim 16, wherein the electrolysis cell E<1> comprises at least one middle chamber K_{M}<13>.

20. The method of claim 16, wherein M_{A} is selected from the is either potassium or sodium.

21. The method of claim 16, wherein the reaction of step (b) is conducted until at least P=10% of the PET used in the reaction of step (b) has been converted.

22. The method of claim 16, wherein the reaction of step (b) is performed at the boiling temperature of the glycol.

23. The method of claim 16, wherein a sufficient amount of solution L₁<21> is used in step (b) so that the total weight of the M_{A} glycolate used in step (b), based on the total weight of the PET used in step (b), is in the range from 0.1% to 100% by weight.

24. method of claim 16, wherein BHET is at least partly separated from M₁ in a further step (c).

25. The method of claim 24, wherein the at least partial separation of BHET from M₁ in step (c) is effected by crystallization and/or distillation.

26. The method of claim 16, wherein the PET is subjected to at least one pretreatment step selected from either a chemical pretreatment step, or a comminution step, before being used in step (b).

27. The method of claim 17, wherein the electrolysis cell E<1> comprises at least one middle chamber K_{M}<13>.

28. The method of claim 27, wherein M_{A} is either potassium or sodium.

29. The method of claim 28, wherein the reaction of step (b) is conducted until at least P=10% of the PET used in step (b) has been converted.

30. The method of claim 29, wherein the reaction of step (b) is performed at the boiling temperature of the glycol.

31. The method of claim 29, wherein a sufficient amount of solution L₁<21> is used in step (b) so that the total weight of the M_{A} glycolate used in step (b), based on the total weight of the PET used in step (b), is in the range from 0.1% to 100% by weight.

32. A method for recycling polyethylene terephthalate, in which BHET is obtained by the method of claim 16 and the BHET thus obtained is polymerized to PET in a step (2).

33. The method of claim 32, wherein the polymerization of BHET to PET in step (2) is conducted at least at the boiling temperature of the glycol.

34. The method of claim 33, wherein the polymerization in step (2) is performed in the presence of a catalyst.

35. The method of claim 34, wherein the catalyst is an antimony compound.
